(19) United States
(12) Patent Application Publication
(10) Pub. No.: US 2022/0042008 A1
(43) Pub. Date: Feb. 10, 2022

(54) COMPOSITIONS OF STREPTAVIDIN-OLIGO CONJUGATES OF pMHC OCCUPANCY

(71) Applicant: MBL International Corp., Woburn, MA (US)

(72) Inventor: Eden Kleinman, Sunnyvale, CA (US)

(21) Appl. No.: 17/415,251

(22) PCT Filed: Dec. 18, 2019
(86) PCT No.: PCT/US19/67211

§ 371 Date: Jun. 17, 2021

Related U.S. Application Data
(60) Provisional application No. 62/781,377, filed on Dec. 18, 2018.

(51) Int. Cl. C12N 15/10; C12Q 1/6804; C12Q 1/686

(52) U.S. Cl. CPC C12N 15/1075; C12Q 1/686; C12Q 1/6804; C12N 15/1065

(57) ABSTRACT

The present disclosure describes pMHC multimer species barcoded with different nucleic acid molecules and the use thereof to determine both the antigen responsiveness and TCR avidity in biological samples and to sequence corresponding T cell transcriptome, T cell proteome, T cell epigenome or the TCR loci.

### RELATED APPLICATIONS

This application claims benefit of and priority to U.S. provisional patent applications 62/781,377, filed Dec. 18, 2018, the contents of which are hereby incorporated by reference.

### BACKGROUND

Barcoded antibodies and barcoded pMHC multimers have recently been developed enabling high-throughput sequencing of cognate cells and bound proteins/peptides¹⁻⁴. Barcoded MHC multimers enable high-throughput multiplexed NGS-based screening of TCR-pMHC binding events with the ability to combine this information with transcriptomic, proteomic and TCR sequence data, potentially on a single cell level. Recent examples demonstrate feasibility and utility of barcoded pMHC multimers^{1,4,5}. However, one limitation is the inability of current technologies to fine-tune pMHC monomer loading per streptavidin to measure TCR-pMHC avidity. There is a need for barcoded pMHC multimers that can assess TCR-pMHC avidity. There is also a need for cost-effective methods to produce barcoded pMHC multimers which avoid chemical conjugation and save on MHC monomer reagents.

### SUMMARY OF THE INVENTION

Provided herein are compositions and methods of producing both covalent and non-covalent conjugated barcoded SA-oligo conjugates (FIG. 1) that can be used as a platform to combine T cell omics analysis in parallel with TCR-pMHC avidity assessment.

In one aspect, the present disclosure provides barcoded pMHC multimer species of varying backbone (e.g. streptavidin) occupancy consisting of at least one biotinylated pMHC molecule non-covalently bound to a streptavidin molecule and at least one nucleic acid molecule covalently or non-covalently bound to the same backbone molecule, wherein nucleic acid may harbor a central barcode region.

In another aspect, the present disclosure provides for barcoding streptavidin with biotinylated oligo using HPLC purification to obtain desired streptavidin occupancy.

In another aspect, the present disclosure provides for barcoding streptavidin with at least one oligo per streptavidin using covalent linkages (e.g. thioether bond or bis-arylhydrazone conjugate bond).

In one aspect, the present disclosure provides pMHC multimers barcoded with at least one nucleic acid molecule which comprises a central barcode region and a poly-A tail.

In another aspect, the present disclosure provides pMHC multimers barcoded with at least one nucleic acid molecule which comprises a central barcode region and a nucleotide sequence complimentary to a TCR constant gene.

In another aspect, the present disclosure provides pMHC multimers barcoded with at least one nucleic acid molecule which comprises a central barcode region and a template switch oligo sequence.

In another aspect, the present disclosure provides methods for making or using the barcoded pMHC multimers disclosed herein.

In one aspect, the present disclosure provides a peptide-Major Histocompatibility Complex (pMHC) barcoded multimer comprising:

at least one tunable pMHC entity, wherein said pMHC entity comprises:
at least one pMHC molecule linked by a backbone molecule: and
at least one nucleic acid molecule per backbone molecule,
   wherein said nucleic acid molecule comprises a covalently or non-covalently linked conjugate.

In some embodiments of the pMHC multimer, the nucleic acid molecule comprises:

a central stretch of pMHC barcoding nucleotides, and

a second stretch of nucleotides with complementarity to a target oligo.

In some embodiments, the pMHC multimer is use in NGS.

In some embodiments of the pMHC multimer, the backbone molecule is a streptavidin.

In some embodiments of the pMHC multimer, the multimer comprises at least one, at least two, at least three, or at least four pMHC entities per backbone molecule.

In some embodiments, the pMHC multimer is use to monitor T cell receptor (TCR)-pMHC avidity.

In some embodiments of the pMHC multimer, the streptavidin is covalently conjugated to the at least one nucleic acid molecule, thereby providing at least four MHC monomers per streptavidin.

In some embodiments of the pMHC multimer, the streptavidin is non-covalently conjugated to the at least one nucleic acid molecule, wherein the nucleic acid molecule is biotinylated, and the at least one biotinylated nucleic acid molecule and streptavidin is complexed in a ratio, wherein the ratio is selected from the group consisting of: 1 streptavidin:1 oligo, 1 streptavidin:2 oligos, and 1 streptavidin:3 oligos.

In some embodiments, the pMHC multimer is produced by the process of HPLC purification.

In some embodiments of the pMHC multimer, the streptavidin is covalently conjugated to the at least one nucleic acid molecule, wherein the nucleic acid molecule comprises a barcode and at least one biotin binding site, wherein said binding site comprises: biotinylated peptides, biotinylated proteins, biotinylated polymers, biotinylated fluorophores, biotinylated cleavable oligos, or biotinylated agents.

In some embodiments of the pMHC multimer, the at least one nucleic acid molecule comprises a 5′ PCR handle region, a central barcode region, optionally a UMI, or optionally a 3′ poly-A tail region of at least 10 consecutive adenines.

In some embodiments of the pMHC multimer, the at least one of the nucleic acid 3′ end tails is comprised of any sequence complementary to a target oligo sequence.

In some embodiments of the pMHC multimer, the at least one nucleic acid molecule is about 10-200 nucleotides in length, or longer.

In another aspect, the present disclosure provides a composition comprising a plurality of subsets of any of the aforementioned pMHC multimer, wherein each subset of pMHC multimer binds a different peptide and has a corresponding barcode region sequence.

In another aspect, the present disclosure provides a method of linking a specific MHC molecule with a corresponding T cell transcriptome, comprising:

a) forming a test sample comprising a plurality of the any of the aforementioned pMHC multimer molecules, T cells, and particles linked to a binding target oligo comprising a 5′ PCR handle, a central cell barcode, UMI, and a bait sequence;

b) forming droplets from the test sample such that each droplet contains no more than one particle, and one T cell bound with one or more pMHC multimer molecules;

c) generating a T cell cDNA library and an pMHC barcode library in each droplet; and

d) sequencing both the T cell mRNA library and the MCH barcode library, thereby linking a specific MHC molecule with the corresponding T cell transcriptome.

In some embodiments of the methods, the bait sequence is 3′ poly-(dT).

In another aspect, the present disclosure provides a multimeric pMHC comprising:

one or more pMHC molecules linked by a backbone molecule; and

at least one nucleic acid molecule linked to said backbone, wherein said nucleic acid molecule comprises a central stretch of nucleic acids (barcode region) designed to be amplified and a nucleotide sequence complimentary to a TCR constant gene.

In some embodiments, the multimeric pMHC comprises a first type of nucleic acid molecule linked to the backbone; and wherein the first type of nucleic acid molecule comprises a central barcode region and a nucleotide sequence complimentary to TCRα or TCRβ constant gene.

In some embodiments, the multimeric pMHC comprises a first and a second type of nucleic acid molecule linked to the backbone; and wherein:

the first type of nucleic acid molecule comprises a central barcode region and a nucleotide sequence complimentary to TCRα constant gene, and

the second type of nucleic acid molecule comprises a central barcode region and a nucleotide sequence complimentary to TCRβ constant gene; and

the barcode regions of the two types of the nucleic acid molecules have the same sequence; and

optionally the UMI sequences for each of the two types of the nucleic acid molecules would be random and thus different from each other although they would be the located in the same region of the respective nucleic acids.

In some embodiments of the multimeric pMHC, the nucleic acid molecule comprises a 5′ PCR handle, a central barcode region, UMI and a 3′ nucleotide sequence complimentary to a TCR constant gene.

In some embodiments of the multimeric pMHC, the nucleic acid molecule comprises a nucleotide sequence complimentary to the 5′ end of the TCR constant gene.

In some embodiments of the multimeric pMHC, the TCR constant gene is TCRα constant gene, TCRβ constant 1 gene, or TCRβ constant 2 gene.

In some embodiments of the multimeric pMHC, the 5′ end and/or the 3′ end nucleic acid molecules are linked to the backbone molecule.

In some embodiments of the multimeric pMHC, the nucleic acid molecule further comprises a unique molecular identifier (UMI) adjacent to the barcode region.

In some embodiments of the multimeric pMHC, the at least one nucleic acid molecule is about 10-200 nucleotides in length, or longer.

In another aspect, the present disclosure provides a composition comprising: a plurality of subsets of any of the aforementioned multimeric pMHC, wherein each subset of multimeric MHC binds a different peptide and has a corresponding barcode region sequence.

In another aspect, the present disclosure provides a method of linking a specific MHC molecule to a corresponding TCRα and/or TCRβ sequences, comprising:

a) providing one or more of any of the aforementioned multimeric pMHC;

b) contacting said multimeric pMHC molecules with T cells;

c) separating T cells bound with the multimeric MHC molecules from those that do not bind the multimeric MHC molecules;

d) lysing the separated T cells;

e) generating a DNA library wherein each DNA molecule comprises a sequence of TCRα and/or TCRβ gene as well as the pMHC barcode; and

f) Sequencing the DNA library, thereby linking the specific pMHC molecule to the corresponding TCRα and/or TCRβ sequences.

In some embodiments of the aforementioned method, the step c) is accomplished by FACS sorting or magnetic bead-based separation.

In some embodiments of the aforementioned method, the multimeric pMHC molecules is directly or indirectly fluorescently labeled.

In some embodiments of the aforementioned method, the T cells bound with barcoded pMHC molecules are bulk sorted in a single collection tube.

In some embodiments of the aforementioned method, the cognate T cells bound with barcoded pMHC molecules are sorted into individual plate wells as single cells.

In another aspect, the present disclosure provides a multimeric pMHC comprising:

one or more pMHC molecules linked by a backbone molecule; and

at least one nucleic acid molecule linked to said backbone, wherein said nucleic acid molecule comprises a central stretch of nucleic acids (barcode region) designed to be amplified, and a template switch oligo sequence.

In some embodiments of the multimeric pMHC, the nucleic acid molecule comprises a 5′ PCR handle, a central barcode region, a UMI and 3′ template switch oligo sequence.

In some embodiments of the multimeric pMHC, the template switch oligo sequence comprises a 3′ stretch of 3 riboguanosines.

In some embodiments of the multimeric pMHC, the at least one nucleic acid molecule is about 10-200 nucleotides in length, or more.

In another aspect, the present disclosure provides a composition comprising: a plurality of subsets of any of the aforementioned multimeric pMHC, wherein each subset of multimeric pMHC binds a different peptide and has a corresponding barcode region sequence.

In another aspect, the present disclosure provides a method of linking a specific pMHC molecule to a corresponding TCRα and/or TCRβ complementary sequences, comprising:

a) forming a test sample comprising a plurality of any of the aforementioned multimeric pMHC molecules, T cells, and beads conjugated to an oligo comprising a 5′ PCR handle, a central cell barcode, UMI and a 3′ nucleotide sequence complementary to a TCR constant gene;

b) forming droplets from the test sample such that each droplet contains no more than one bead, and one T cell bound with one or more multimeric MHC molecules;

c) generating a DNA library wherein each DNA molecule comprises a sequence of TCRα and/or TCRβ gene as well as the pMHC barcode; and

d) sequencing the DNA library, thereby linking a specific pMHC molecule to a corresponding TCRα and/or TCRβ sequences.

In some embodiments of the method, the bead is selected from hydrogel bead, hard bead and dissolvable bead.

In some embodiments of the method, the bead is conjugated to two oligos, wherein the first oligo comprises a 5′ PCR handle, a central cell barcode, UMI and a 3′ nucleotide sequence complementary to a TCRα constant gene; the second oligo comprises a 5′ PCR handle, a central cell barcode, UMI and a 3′ nucleotide sequence complementary to a TCRβ constant gene; and the central cell barcodes for the two oligos have the same sequence.

In some embodiments of the method, the DNA library generation step c) comprises reverse transcription of TCR mRNA using MMLV reverse transcriptase.

In some embodiments of any of the aforementioned multimeric pMHC and methods, the PCR handle enables the library preparation of the barcode sequence.

In some embodiments of any of the aforementioned multimeric pMHC and methods, the PCR handle may have the i7 adapter sequence.

In some embodiments of any of the aforementioned multimeric pMHC and methods, the barcode region comprises at least 4 nucleotides.

In some embodiments of any of the aforementioned multimeric pMHC and methods, the pMHC molecule is linked to the backbone via a streptavidin-biotin binding, via the MHC heavy chain, or via the MHC light chain (β2M).

In some embodiments of any of the aforementioned multimeric pMHC and methods, the MHC molecule is linked to the backbone via a streptavidin-biotin binding.

In some embodiments of any of the aforementioned multimeric pMHC and methods, the multimeric pMHC comprises at least one MHC molecule.

In some embodiments of any of the aforementioned multimeric pMHC and methods, the at least one nucleic acid molecule further comprises chemical modifications.

In some embodiments of any of the aforementioned multimeric pMHC and methods, the 5′ or 3′ end of the at least one nucleic acid molecule is attached to an amino group via a spacer.

In some embodiments of any of the aforementioned multimeric pMHC and methods, the spacer can be a 6-carbon spacer or a 12-carbon spacer.

In some embodiments of any of the aforementioned multimeric pMHC and methods, the at least one nucleic acid molecule comprises phosphorothioated nucleotides at the 5′ end and/or 3′ end.

In some embodiments of any of the aforementioned multimeric pMHC and methods, the linkage between the at least one nucleic acid molecule and the backbone molecule allow for inducible dissociation of the nucleic acid molecules.

In some embodiments of any of the aforementioned multimeric pMHC and methods, the at least one nucleic acid molecule is linked to the backbone molecule via a covalent or non-covalent bond.

In some embodiments of any of the aforementioned multimeric pMHC and methods, the covalent bond is a thioether.

In some embodiments of any of the aforementioned multimeric pMHC and methods, the at least one nucleic acid molecule is linked to the backbone molecule via an inducibly cleavable bond

In some embodiments of any of the aforementioned multimeric pMHC and methods, the inducibly cleavable bond is photocleavable, or comprises disulfide linkages.

In some embodiments of any of the aforementioned multimeric pMHC and methods, the MHC molecule is MHC class I and/or MHC class II monomer.

In some embodiments of any of the aforementioned multimeric pMHC and methods, the MHC molecule is complexed with a peptide.

In some embodiments of any of the aforementioned multimeric pMHC and methods, the MHC molecule is biotinylated.

In some embodiments of any of the aforementioned multimeric pMHC and methods, the backbone further comprises one or more labels selected from the group consisting of fluorescent labels, His-tags, and metal-ion tags.

In some embodiments of any of the aforementioned multimeric pMHC and methods, the backbone is directly conjugated with the fluorescent labels.

In some embodiments of any of the aforementioned multimeric pMHC and methods, the fluorescent label is a fluorophore-tagged oligo.

In some embodiments of any of the aforementioned multimeric pMHC and methods, the fluorophore-tagged oligo is complementary to the nucleic acid molecule linked to the backbone.

In some embodiments of any of the aforementioned multimeric pMHC and methods, the backbone is labeled with a fluorophore labeled anti-streptavidin antibody.

In some embodiments of any of the aforementioned multimeric pMHC and methods, the fluorophore is a fluorescent protein, fluorescent dye, or quantum dot.

In some embodiments of any of the aforementioned multimeric pMHC and methods, the at least one nucleic acid molecule comprises a nucleic acid molecule selected from the group consisting of DNA, RNA, artificial nucleotides, PNA, and LNA.

In some embodiments of any of the aforementioned multimeric pMHC and methods, the multimeric pMHC binds cognate T cells.

In some embodiments of any of the aforementioned multimeric pMHC and methods, the multimeric pMHC is compatible with flow cytometric applications.

In some embodiments of any of the aforementioned multimeric pMHC and methods, the flow cytometric application is single cell or bulk cell fluorescence-activated cell sorting (FACS).

In some embodiments of any of the aforementioned multimeric pMHC and methods, the multimeric pMHC is compatible with NGS-based applications.

In some embodiments of any of the aforementioned multimeric pMHC and methods, the NGS-based application is droplet-based single cell sequencing.

In another aspect, the present disclosure provides a method for detecting antigen responsive cells in a sample comprising:

a) providing one or more of any of the aforementioned multimeric pMHC;

b) contacting said multimeric pMHC molecules with said sample; and

c) detecting binding of the multimeric pMHC molecules to said antigen responsive cells, thereby detecting cells responsive to an antigen present in the MHC molecules, wherein said binding is detected by amplifying the barcode region of said nucleic acid molecule linked to the one or more MHC molecules through the backbone molecule.

In some embodiments of the method, the sample is selected from the group consisting of blood sample, a peripheral blood sample, a blood derived sample, a tissue sample, a body fluid, spinal fluid, and saliva.

In some embodiments of the method, the sample is obtained from a mammal.

In some embodiments of the method, the method further comprises cell selection by a method selected from the group consisting of flow cytometry, FACS, magnetic-bead based selection, size-exclusion, gradient centrifugation, column attachment, and gel-filtration.

In some embodiments of the method, the amplification is PCR.

In some embodiments of the method, the detection of barcode regions of the nucleic acid molecule includes sequencing of the barcode regions or detection of the barcode region by qPCR.

### BRIEF DESCRIPTION OF THE DRAWINGS

FIG. 1 depicts the general structure of a modified oligo for conjugation to streptavidin for making barcoded pMHC multimers. Purple bar represents the oligo and the green star represents a 5′ modification. Below is the sequence of an oligo used in covalent conjugations. The 5′ end of the oligo is an amino group with a 12-carbon spacer. A 6-carbon spacer can also be used. Alternative oligo modifications can be used instead of an amino group including, but not limited to, a thiol group or a biotin group. The 5′ biotin group can be used for non-covalent linkage to streptavidin. These oligo modifications can also be incorporated on the 3′ end of the oligo. The PCR handle enables library preparation of barcode sequence. In this particular case, the PCR handle is the i7 adapter sequence. Tetramer barcode sequence follows after the PCR handle and is the sequence that corresponds to a given pMHC complex. In this sequence the pMHC barcode is made of 6 nucleotides which allow up to 4096 tetramer barcode possibilities. Longer pMHC barcode sequences can be used for increased throughput. At the 3′ end of the oligo is the poly-A tail which can bind to a target sequence, in this case poly(dT)VN. For this oligo, 25 adenines are used although longer adenine stretches can be used. The B nucleotide (G, C or T) at the 5′ end of the poly-A tail enables binding to the V (G, C, or A) nucleotide at the second to last 3′ nucleotide of the poly(dT)VN sequence. If the N nucleotide (any base) of the bait sequence is a C then it binds complementary with the oligo sequence depicted. Optional phosphorothioated DNA bases on the 3′ end of the oligo provide protection from exonuclease activity. Modified bases can be placed at any position.

FIG. 2 shows the conjugation of barcoding oligo to streptavidin with disulfide bridge in between. In the example shown, conjugation can be performed using the Solulink Protein-Oligonucleotide Conjugation Kit (TriLink Biotech). Streptavidin (in this case from Prozyme) is modified with succinimidyl-6-hydrazino-nicotinamide (S-HyNic). 5′-amino-modified oligo is modified with succinimidyl-4-formylbenzamide analog (S-SS-4FB). Modified streptavidin and modified oligo are combined producing a barcoded streptavidin. An alternative methodology includes conjugating oligos to streptavidin with an intervening photocleavable linker (not depicted).

FIG. 3 shows that the DNA barcoding oligo dissociates from streptavidin under denaturing conditions. 66-mer oligo conjugated to streptavidin with an intervening disulfide bridge is run on a SYBR-Safe agarose gel (Lane 1a). Excess oligo is indicated below the 100 bp ladder band. In lane 2a, the same amount of barcoded streptavidin as in Lane 1a is incubated with beta-mercaptoethanol for 20 minutes prior to loading onto gel. The band representing the barcoded streptavidin is severely diminished. The same gel was stained with Coomassie blue to visualize protein. In Lane 1b, barcoded streptavidin is evident whereas most of the protein in Lane 2b is shifted to the top of the gel. Without the negative charge of the oligo, bare streptavidin migrates much slower.

FIG. 4 shows that barcoded streptavidin retains ability to bind biotin. Equal amounts of barcoded streptavidin were incubated with either biotin magnetic beads (Lane 1a) or streptavidin magnetic beads (Lane 2a). Equivalent amount of biotin magnetic beads or streptavidin magnetic beads were used for this experiment. Samples were subjected to a magnetic separator to separate bound versus unbound barcoded streptavidin. All unbound fraction from either reaction was harvested and run on a SYBR-Safe agarose gel. The same gel was stained with Coomassie blue to visualize protein (Lane 1b and 2b). Depletion of barcoded streptavidin (Lanes 1a and 1b) indicates biotin binding. Both biotin and streptavidin magnetic beads were purchased from RayBiotech Inc.

FIG. 5 shows barcoded pMHC multimer variants. Far left barcoded pMHC multimer is composed of a streptavidin-oligo conjugate derived from covalent conjugation, after which biotinylated pMHC monomers are used to tetramerize according to established techniques⁶. This covalent conjugation can derive from many conjugation chemistries including but not limited to thioether bond formation.

All other conjugates utilize non-covalent linkages between biotinylated oligo and streptavidin. Second from left, barcoded pMHC multimers are composed of purified streptavidin-oligo conjugates at a ratio of 1:1 (1b), which are then pMHC trimerized. Third from left, barcoded pMHC multimers are composed of purified streptavidin-oligo conjugates at a ratio of 1:2 (2b), which are then pMHC dimerized. At the far right, barcoded pMHC multimer is composed of purified streptavidin-oligo conjugates at a ratio of 1:3, which are then combined with pMHC monomers to give a streptavidin:monomer molar ratio of 1:1. The different colored oligos represent different barcode sequences for each multimer species. Bottom triangle depicts the predicted TCR-pMHC avidity of each multimerized conjugate whereby 4 monomers>3 monomers>2 monomers>1 monomer.

FIG. 6 contains four panels, FIG. 6A-6D, depicting methodologies of barcoded pMHC multimer detection. In FIG. 6A, fluorophore-containing streptavidin (e.g. PE-streptavidin) is used as a starting point for further conjugation to oligo. In FIG. 6B, a fluorophore-tagged oligo, complementary to the conjugated oligo, is annealed to the pMHC barcoding oligo. The length of this annealing oligo can be varied. In FIG. 6C, fluorophore labeled anti-streptavidin antibody is used to detect barcoded pMHC multimers. Chemical species of fluorophores used can include but are not limited to fluorescent dyes and quantum dots. Barcoded pMHC multimer species wherein the oligos are non-covalently linked can also be detected using these methods. FIG. 6D illustrates the possibility of separately labeling different pMHC multimer species (containing the same peptide) with different fluorescent dye containing anti-streptavidin antibodies. Any of the three fluorescent labeling techniques in FIG. 6A-6C could be applied. In this way, different TCRs can be screened for their avidity towards this particular pMHC protein complex using flow cytometry. In this particular situation, the 4 monomer pMHC multimer (tetramer) does not require an oligo covalently conjugated and the pMHC multimer species utilizing non-covalently bound oligos do not require differing oligo barcode sequences if all that is required is for oligo to block biotin binding site(s). Other biotinylated molecules could theoretically be used to block biotin binding sites as outlined in FIG. 14.

FIG. 7 illustrates an application example using barcoded pMHC multimers for immuno-epitope avidity monitoring. A tumor-derived mutant gene-encoded protein is peptide tiled for TCR sequencing studies. In this depiction, two overlapping peptide amino acid sequences (depicted by numbers) from that same mutant protein, are individually complexed with biotinylated MHC (same MHC allele) and then with barcoded streptavidin conjugates as shown. Oligo sequences for all conjugate types, regardless of pMHC, will contain a unique barcode sequence that distinguishes them (represented by colored lines attached to streptavidin). Barcoded pMHC multimers are mixed together with sample. TCR alpha and beta sequences are obtained via single-cell RNA sequencing and paired with cognate pMHC multimer type. As an example, one study using these conjugates could determine whether the green peptide-MHC complex or the red peptide-MHC complex has a higher avidity for a mutually bound TCR sequence.

FIG. 8 illustrates another application example of barcoded pMHC multimer immuno-epitope avidity monitoring. In this hypothetical example, a vial contains a defined mixture of barcoded pMHC multimer species composed of streptavidin:oligo ratios 1:1, 1:2 or 1:3, which can also be defined as a streptavidin:pMHC monomer molar ratios of 1:3, 1:2 and 1:1, respectively. Oligos for conjugate species will contain a unique barcode sequence which will distinguish them (different colored sequences). Because TCR-pMHC avidity is purported to be diminished within the tumor microenvironment, it is predicted that these barcoded pMHC multimer conjugate species will be able to demonstrate increased or decreased TCR avidity during the course of therapy, e.g. Immune Checkpoint Blockade (ICB) therapy. In the example given, post-ICB treated T cells increase in avidity with cognate pMHC complex as evidenced by higher read count of multimers containing 1 and 2 pMHC monomers.

FIG. 9 depicts generation of non-covalently bound barcoded pMHC multimer for detection via NGS. Biotinylated oligo (containing pMHC multimer-specific barcode) is mixed with streptavidin (or streptavidin-fluorophore conjugate) at a ratio of 0.5-1.0 oligo to 1 streptavidin. 1:1 SA:oligo conjugates are precisely purified via HPLC such that unreacted streptavidin, unreacted biotinylated oligo and conjugates of differing SA:oligo ratios are removed. Conjugates are then multimerized with biotinylated pMHC monomers. The final products are barcoded pMHC trimers where each streptavidin has one oligo and three pMHC monomers. The same concept is used when making conjugates consisting of streptavidin:oligo ratios of 1:2 and 1:3.

FIG. 10 shows that barcoded pMHC multimers of differing monomer ratios can similarly detect T cells when used at a high enough concentration and when detecting TCRs of moderate avidity. Streptavidin-oligo conjugates were manufactured by linking biotinylated oligo to streptavidin at SA:oligos ratios of 1:1 or 1:2. Streptavidin alone was used as a control, which had 4 pMHC monomers per streptavidin (tetramer) as depicted in the figure legend at the bottom. 1:1 SA:oligo conjugates contained 3 monomers whereas 1:2 SA:oligo conjugates contained 2 monomers. For the purposes of this experiment, the oligo for all barcoded conjugates was composed of the same 69mer nucleotide sequence. The left three columns depict multimer staining using HLA-A*11:01 MHC I complexed with CMV pp65 peptide which was then multimerized with either streptavidin, streptavidin with one oligo or streptavidin with two oligos. The middle three columns depict multimer staining using HLA-A*11:01 MHC I complexed with EBV 399-408 peptide. The right three columns depict multimer staining using HLA-A*11:01 MHC I complexed with EBV 416-424 peptide. The amount of streptavidin used in each stain is indicated on the far left side of the row. The percent multimer positive staining is indicated in each box. Previously peptide expanded cells were utilized for staining with anti-CD3, anti-CD8, live/dead dye as well as non-fluorophore barcoded pMHC multimers (or tetramer). Secondary stain was composed of anti-streptavidin-PE to be able to detect pMHC multimers. Flow cytometry gate legend is shown at top of figure.

FIG. 11 illustrates that barcoded pMHC multimers of differing oligo and monomer ratios can distinguish pMHC-TCR avidities. Streptavidin-oligo conjugates were manufactured by either covalently linking oligo to streptavidin via a covalent bond (thioether bond) or by linking biotinylated oligo to streptavidin at various SA:oligos ratios. For the purposes of this experiment, oligos for all conjugates were composed of the same 46mer nucleotide sequence. Covalent conjugates indicate one streptavidin molecule conjugated to one oligo with all four biotin binding sites available. 1b conjugates indicate one streptavidin linked to one biotinylated oligo with three biotin binding sites remaining. 2b conjugates indicate one streptavidin linked to two biotinylated oligos with two biotin binding sites remaining. 3b conjugates indicate one streptavidin linked to three biotinylated oligos with one biotin binding site remaining. H-2Kb biotinylated monomers (MBLI) were complexed with either high affinity SIINFEKL peptide or low affinity SIIVFEKL peptide⁸. Affinity in this case refers to interaction strength between pMHC complex to transgenic OT-I TCR, not of peptide interaction with MHC. Avidity in this case refers to the combined affinity when multiple pMHC are involved in TCR binding assessments.

pMHC molecules were complexed with streptavidin-oligo conjugates to generate barcoded pMHC multimers. OT-I splenocytes were stained with the various barcoded H-2Kb barcoded pMHC multimer species at various SA amounts (0.25 ug first row, 0.1 ug second row, 0.025 ug third row). Covalent conjugates were pMHC tetramerized using a molar ratio of 1 SA to 4 monomers. 1b conjugates were pMHC trimerized using a molar ratio of 1 SA to 3 monomers. 2b conjugates were pMHC dimerized using a molar ratio of 1 SA to 2 monomers. 3b conjugates were pMHC conjugated using a molar ratio of 1 SA to 1 monomer. Cells were simultaneously stained with anti-CD3, anti-CD8, live/dead dye and respective non-fluorophore barcoded pMHC multimer species. Secondary stain was composed of anti-streptavidin-PE to detect pMHC multimers. Flow cytometry gate legend is shown at top of figure. The percent pMHC multimer positive staining is indicated in each box, at either low, medium or high intensity.

FIG. 12 illustrates a hypothetical experimental result using barcoded pMHC multimers of differing pMHC occupancy to distinguish pMHC-to-TCR avidities. SIINFEKL and SIIVFEKL peptide-derived barcoded pMHC multimers are depicted mixed with OT-I splenocytes (left side) in a hypothetical experimental setting. The ratio of each multimer species can be adjusted based on experimental goals. Alternatively, an aliquot of cells can be separately incubated with either SIINFEKL or SIIVFEKL barcoded pMHC multimers. Each multimer species would harbor a unique barcode sequence to distinguish itself from other multimer species. FACS sorting can be used prior to droplet-based single cell sequencing or other single cell sequencing platforms (middle). On the right side, predicted barcode read frequencies. In this case, high affinity/avidity SIINFEKL-based pMHC barcode reads would predominate at every monomer species whereas low affinity/avidity SIIVFEKL-based pMHC barcode reads would likely only show up at 4 monomer species. For this particular situation, use of just the 4 monomer-based pMHC multimer species for each peptide (SIINFEKL vs SIIVFEKL) would be sufficient to determine which pMHC complex is of higher avidity towards this particular TCR. However, in cases where the avidity variance is more subtle and where different TCRs are involved, use of pMHC barcoded species can potentially reveal differences in avidity.

FIG. 13 shows that barcoded pMHC multimers can be generated with directly conjugated fluorophore. Streptavidin-oligo conjugates were manufactured by either covalently linking oligo streptavidin via a thioether bond (Coy) or by linking biotinylated oligo to streptavidin at a ratio of 1 SA:1 oligo (1b). In addition, a fluorophore (Alexa Fluor 647) containing SA-oligo conjugate was manufactured. In this case, AF647 was first directly conjugated to streptavidin, after which oligo was directly conjugated to streptavidin. For the purposes of this experiment, conjugates were composed of the same 69mer nucleotide sequence. Covalent conjugates indicate one streptavidin molecule conjugated to one oligo with all four biotin binding sites available. 1b conjugates indicate one streptavidin conjugated to one biotinylated oligo with three biotin binding sites remaining. Conjugates were multimerized with either biotinylated CMV pp65 NLVPMVATV peptide containing HLA-A*02:01 monomers or Negative control HLA-A*02:01 monomers (MBLI) at the ratios indicated. Human HLA-A*02:01 PBMCs that were previously expanded with CMV pp65 peptide (NLVPMVATV) were used for staining with conjugates. Cells were stained with anti-CD3, anti-CD8, live/dead dye and barcoded pMHC multimers. Middle column sample was stained with MBLI commercial CMV pp65 HLA-A*02:01 PE tetramer as a control. Samples containing conjugates devoid of fluorophores were secondarily stained with anti-SA-PE. Flow cytometry gate legend is shown at top of figure. The percent pMHC multimer positive staining is indicated in each box.

FIG. 14 shows alternative methods to create barcoded pMHC multimer species. All conjugate species depicted harbor one oligo per streptavidin via a covalent linkage (e.g. thioether bond). In an actual experimental setting, each conjugate species (4, 3, 2 or 1 monomer(s)) would carry a unique barcode sequence depicted by a different color. Biotinylated peptides, biotinylated proteins, biotinylated lipids, biotinylated fluorophores, biotinylated polymers or cleavable biotinylated oligos (all possibilities generically depicted in purple) are incubated with SA-oligo conjugate in an optimized ratio and HPLC-purified to the desired biotin binding occupancy. These purified conjugates would then be multimerized with biotinylated pMHC monomers leading to full occupancy of biotin binding sites per streptavidin. Bottom triangle depicts the predicted TCR-pMHC avidity of each multimerized conjugate whereby 4 monomers>3 monomers>2 monomers>1 monomer.

FIG. 15 depicts bulk pMHC multimer barcode sequencing for immune-epitope dominant analysis. In this embodiment, barcoded pMHC multimers (stars with different colored lines) are quantified from cells with or without the use of enrichment such as magnetic bead separation or FACS. In the example shown, 5 different gp100 peptides have been made into separate barcoded pMHC multimers where each gp100 peptide/MHC complex is associated with a unique multimer barcode (represented by different colors). Barcoded pMHC multimers can utilize either covalent or non-covalently linked oligos. Oligos would be processed for library preparation and sequencing.

For applications requiring small barcoded oligo libraries (e.g. 8 or less), pMHC multimer barcode quantification can be ascertained without the use of sequencing. Fluorescently labeled oligos that are complementary to a region of the pMHC multimer can be pre-annealed prior to incubation with cells. Each barcoded pMHC multimer type would be annealed to a distinct fluorophore containing oligo prior to incubation with cells and subsequent flow cytometry. For example, pMHC multimers containing the gp100#1 peptide and associated oligo sequence would be pre-annealed to complimentary AlexaFluor488-oligos, pMHC multimers containing gp100#2 peptide and associated oligo sequence would be pre-annealed to complimentary AlexaFluor532-oligos, etc.

FIG. 16 illustrates use of barcoded pMHC multimers with NGS-based single cell sequencing platforms. Barcoded pMHC multimer (covalently bonded oligo in this example) bound to target cells are captured in a single droplet. Most individual droplets contain one T-cell/barcoded pMHC complex along with a particle containing target oligos. In this example the barcoding oligo contains a poly-A. Both the oligo and T cell mRNA are reverse-transcribed for further library preparation. Optionally, a cleavable bond (UV or disulfide) in-between the oligo and streptavidin can be introduced during the conjugation of oligo to streptavidin. In particular, disulfide bonds will dissociate in the droplet due to the reducing environment of the lysis buffer.

FIG. 17 contains eight panels, FIG. 17A-17H, showing TCR loci targeting barcoded pMHC multimers to specifically sequence TCR and pMHC complex reads. An alternative to oligo designs in bulk sequencing and droplet-based NGS platforms is to have an oligo that targets desired endogenous mRNA transcripts while also maintaining the identity of the pMHC complex. Depicted are various designs for barcoded pMHC multimers to specifically interrogate TCR clonality or single cell TCR repertoire along with corresponding pMHC identity. These designs allow for sequencing of TCR transcripts along with pMHC barcode in one read. The PCR handle involves the area where PCR primer will bind for amplification. Barcode (BC) represents the nucleotide sequence identifying the pMHC complex. TCR complementary sequence (TCR compl) is complementary to the TCR constant gene. TCR complementary sequences will serve as a primer during reverse transcription.

In FIG. 17A, barcoding oligo is covalently linked to streptavidin to make a barcoded tetramer. This barcoded tetramer contains a 3′ sequence complementary to the TCRβ constant region gene. The TCRβ constant gene oligo sequence is complementary to both the TCRβ constant 1 gene and TCRβ constant 2 gene as there is significant sequence similarity between the two constant genes at the 5′ end (closest to respective J genes). The purified barcoded streptavidin conjugate would then be tetramerized with pMHC. Unique molecular identifiers (UMIs) can also be placed adjacent to the tetramer barcode for downstream PCR deduplication and thus more accurate pMHC binding quantification. TCRβ targeting oligos alone can be used to study TCR clonality. Alternatively, TCRα constant gene targeting oligos can be used alone (not depicted).

In FIG. 17B, the purified streptavidin-oligo conjugate from A is further modified to include a covalently attached oligo targeting the TCRα constant gene. This construct would be suitable for single cell sequencing platforms to derive a complete TCRα/β sequence (same for FIGS. 17D-H). This dual oligo streptavidin conjugate would then be tetramerized.

In FIG. 17C, a TCRβ targeting biotinylated oligo is non-covalently attached to streptavidin, purified and then trimerized with biotinylated pMHC monomers.

In FIG. 17D, the purified TCRβ conjugate from C is further modified by non-covalently attaching a TCRα targeting biotinylated oligo, purified and then dimerized with biotinylated pMHC monomers.

In FIG. 17E, a singular oligo is used that contains both TCRβ and TCRα targeting sequences and is biotinylated on both ends. Desired conjugates would be purified and dimerized with biotinylated pMHC monomers. An alternative oligo design for this would include a cleavable (UV or disulfide) bond in between the two halves.

In FIG. 17F, a singular oligo biotinylated on both ends is used that contains both TCRβ and TCRα targeting sequences. Streptavidin-oligo conjugates of the correct size are purified and 2× trimerized with biotinylated pMHC monomers, thus giving a total of 6 pMHC monomers per oligo. As in FIG. 17E, this design can be further enhanced to include a cleavable (UV or disulfide) bond in between the two halves.

In FIG. 17G, a singular oligo containing both TCRβ and TCRα targeting sequences is covalently attached to streptavidin. Purified conjugates are tetramerized with biotinylated pMHC monomers. As in FIG. 17E and FIG. 17F, this conjugate could include a cleavable bond.

In FIG. 17H, a singular biotinylated oligo containing both TCRβ and TCRα targeting sequences is non-covalently attached to streptavidin. Purified conjugates are trimerized with biotinylated pMHC monomers. As in FIGS. 17E-G, this conjugate could include a cleavable bond.

FIG. 18 shows TCR loci targeting barcoded pMHC multimers used in FACS sorted single cell TCR clonality or paired TCRα/β sequencing. For the purposes of illustration, only several designs are shown but any barcoded pMHC multimer design from the previous figure could potentially be used.

Depicted on the left side are barcoded tetramers containing TCRβ constant gene targeting oligos. Alternatively, TCRα constant gene targeting oligos can be used or both can be used together with designs described in the preceding figure. These complementary TCR α/β oligo sequences will serve as reverse transcription primers for endogenous TCRα/β mRNA transcripts. To employ TCR targeting barcoded pMHC multimers in cell sorting applications, fluorescence can be incorporated as described in FIG. 6. T cells are first single cell sorted and in-well reverse transcribed, then bulk processed in a single tube for clonality studies. T cell #1 is bound by barcoded pMHC multimers containing the barcode sequence #1 while T cell #2 is bound by barcoded pMHC multimer containing barcode sequence #2. On the right side, barcoded pMHC multimers harbor both TCR a and (3 targeting sequences as described in the preceding figure. T cells are single cell sorted and processed in individual plate wells, in this case a 96-well plate.

Clonality studies allow researchers to understand the breadth of TCR usage for a given pMHC complex. The downside to bulk sequencing if both TCRα and TCRβ barcoding oligos are used, is that TCRα and TCRβ sequences cannot be paired. To overcome this challenge, T cells can be processed as shown on the right side which allows pairing of TCRα and TCRβ sequences.

Library preparation strategies are described below and are expanded upon in proceeding Figures:
i) Single sorted T cells are reverse transcribed and subsequently processed in bulk. RNase treatment is followed by bridge adapter ligation. The bridge adapter is common to all reverse-transcribed genes. Subsequent PCR amplifications yields final library prep.
ii) Single sorted cells are reverse transcribed by SMARTScribe RT (Clontech). SMARTer first-strand synthesis and RT template switching yield 5′-RACE Ready cDNA. Subsequent PCR amplifications in bulk yields final library prep.
iii) Single cell sorted T cells are lysed and undergo in-well RT, in-well RNase treatment followed by in-well bridge adapter ligation. The bridge adapter upstream sequence is common to all wells but every well receives a bridge adapter with a unique cell barcode at the site closest to the TCR sequence. The major advantage of this methodology is that TCRα and TCRβ sequences arising from the same cell can be paired so that the complete TCR sequence of individual T cells can be ascertained. Ligated transcripts are pooled for subsequent PCR amplification using common primers yielding final library prep.
iv) Single sorted T cells are lysed and undergo in-well SMARTScribe RT (Clontech). SMARTer first-strand synthesis and RT template switching yield 5′-RACE Ready cDNA. To individually barcode each well, in-well PCR amplification is performed using forward primers complementary to SMARTerIIA oligo with unique cell barcode sequences upstream of the SMARTerIIA binding sequence. This will distinguish transcripts from individual wells. At the 5′ end of the forward primer, a common priming site (i.e. i5 sequence) allows sample pooling. Pooled PCR amplification using common primers yields final library prep. As with iii, the major advantage of this methodology is that TCRα and TCRβ sequences arising from the same cell can be paired.

FIG. 19 illustrates TCR targeting barcoded pMHC multimer library preparation for clonality studies using bridge adapters. As described in FIG. 18(i), barcoded pMHC multimers bound by cognate T cells are first single cell sorted, reverse transcribed and then bulk processed. For display, both TCRβ and TCRα derived transcripts are shown although only one needs to be used for clonality studies. Use of fluorophore tracking strategies described in FIG. 6 are needed for FACS sorting. In-well RT is followed by RNase treatment and then bridge adapter ligation. The bridge adapter (i5 sequence) for bulk sequencing is identical for all cells and contains a 5′ phosphate on the bottom oligo of the bridge adapter that is necessary for ligation to the 3′ end of the newly reverse-transcribed TCR transcript. The bridge adapter also contains 6 random nucleotides on the 3′ end of the non-ligating strand to enhance stability of adapter binding⁹. Following ligation, PCR amplification with common primers yields final library prep.

FIG. 20 illustrates TCR targeting barcoded pMHC multimer library preparation for bulk sequencing using SMARTScribe enzyme (Clontech). As described in FIG. 18(ii), barcoded pMHC multimers bound by cognate T cells are first single cell sorted, reverse transcribed and then bulk processed. For display, both TCRβ and TCRα derived transcripts are shown although only one needs to be used for clonality studies. Use of fluorophore tracking strategies described in FIG. 6 are needed for FACS sorting. In-well RT with SMARTScribe enzyme adds the SMARTerIIA oligo sequence via template switching to the 3′ end of the newly reverse-transcribed transcript. Following template switching, bulk PCR amplification with common primers yields final library prep.

FIG. 21 illustrates TCR targeting barcoded pMHC multimer library preparation for single cell paired TCRα/β sequencing using bridge adapters. As described in FIG. 18(iii), barcoded pMHC multimers bound by cognate T cells are single cell sorted into individual wells. Use of fluorophore tracking strategies described in FIG. 6 are needed for FACS. Sorted cells are lysed and undergo in-well RT, in-well RNase treatment, and then in-well bridge adapter ligation (for simplicity only a single TCR transcript is shown at the ligation step). The bridge adapter (e.g. i5 sequence) for each well contains a unique cell barcode that will enable pairing of TCRα/β sequences. In this illustration, 00001 represents the cell barcode for this particular well. A 5′ phosphate on the ligating strand of the bridge adapter enables ligation to the 3′ end of the newly reverse-transcribed TCR transcripts. The bridge adapter also contains 6 random nucleotides on the 3′ end of the non-ligating strand to enhance stability of adapter binding. Following ligation, samples are pooled for PCR amplification with common primers yielding the final library prep.

FIG. 22 illustrates TCR targeting barcoded pMHC multimer library preparation for single cell sequencing using SMARTScribe enzyme. As described in FIG. 18(iv), barcoded pMHC multimers bound by cognate T cells are single cell sorted into individual wells. Use of fluorophore tracking strategies described in FIG. 6 are needed for FACS. Sorted single cells are lysed and undergo in-well RT with SMARTScribe enzyme (Clontech) which adds the SMARTerIIA oligo sequence via template switching to the 3′ end of the newly reverse-transcribed transcript. Following this, in-well PCR amplification (only a single TCR transcript shown for simplicity) is performed using forward primers that contain both SMARTerIIA binding sequence as well as a cell barcode sequence (unique to every well) and a common i5 sequence at the 5′ end. In this illustration, the cell barcode is represented by 00001. Use of forward primers that contain unique cell barcodes for every well enable pairing of TCRα/β sequences. Samples are pooled for further PCR amplification yielding final library prep.

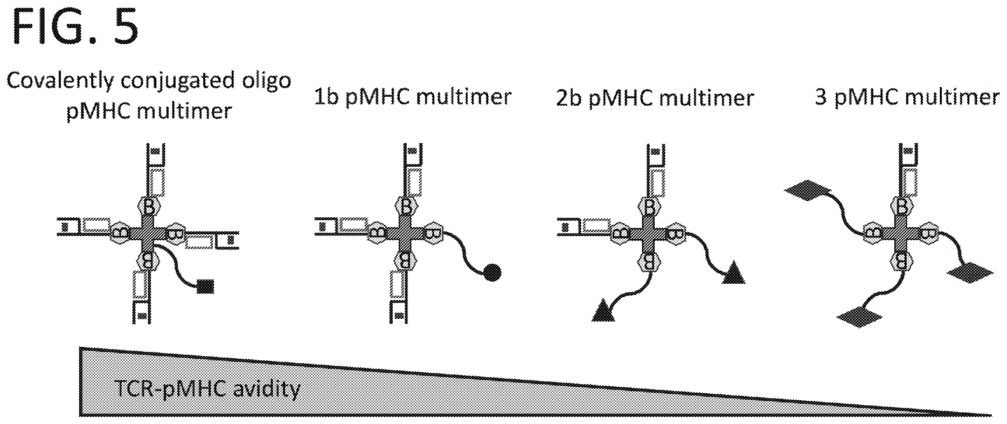

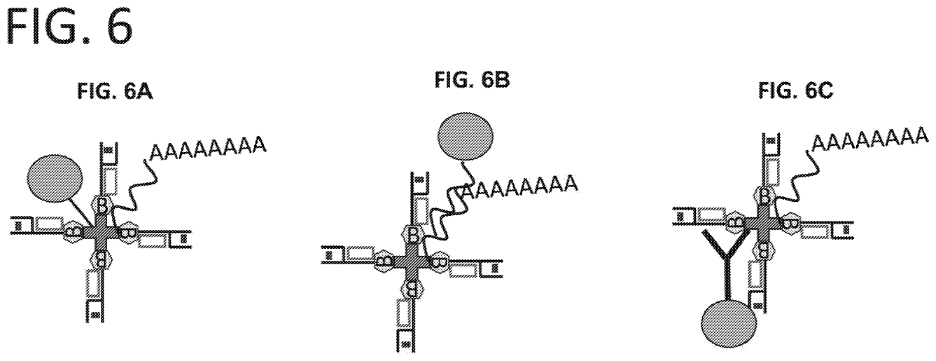

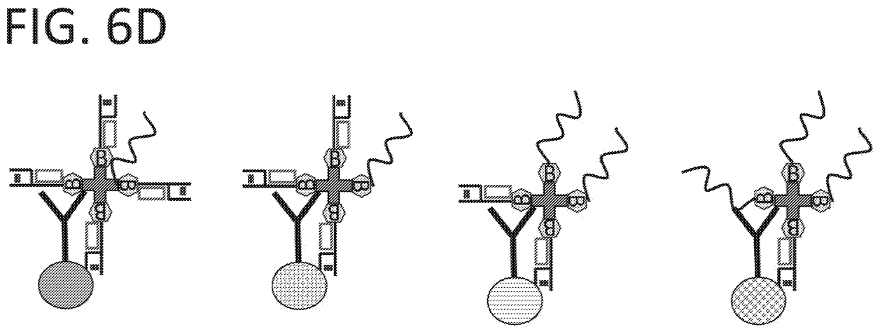

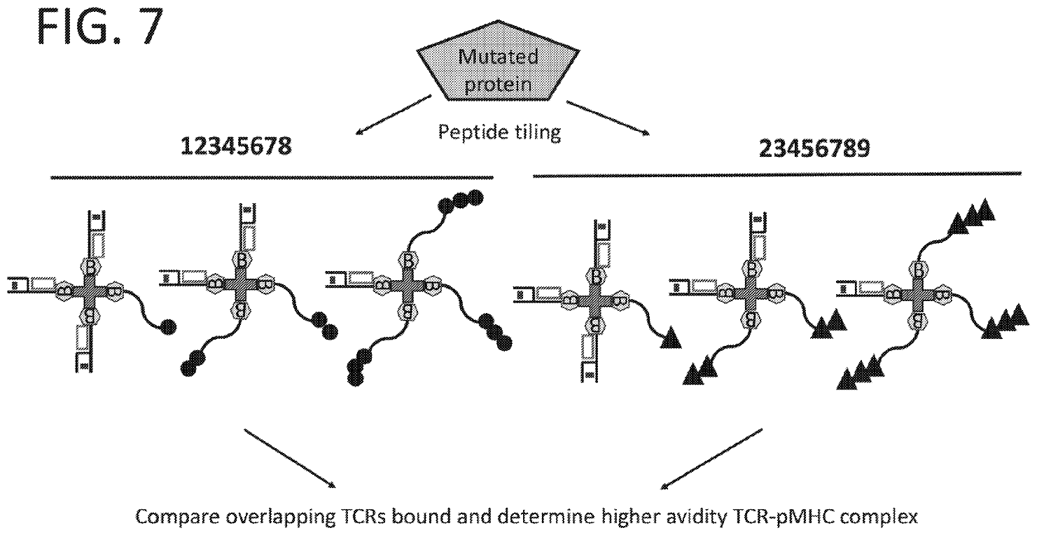

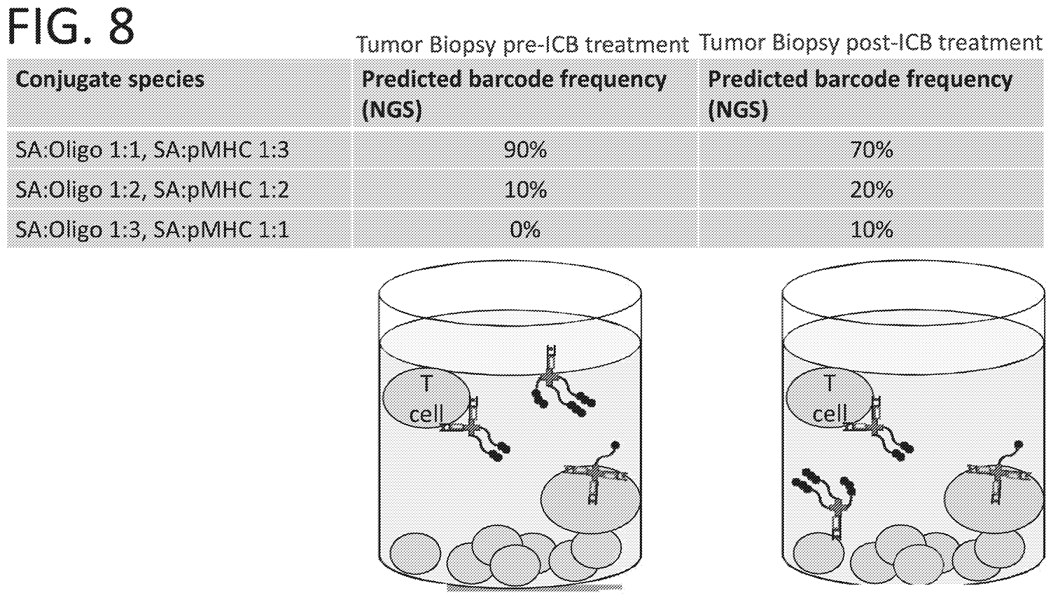

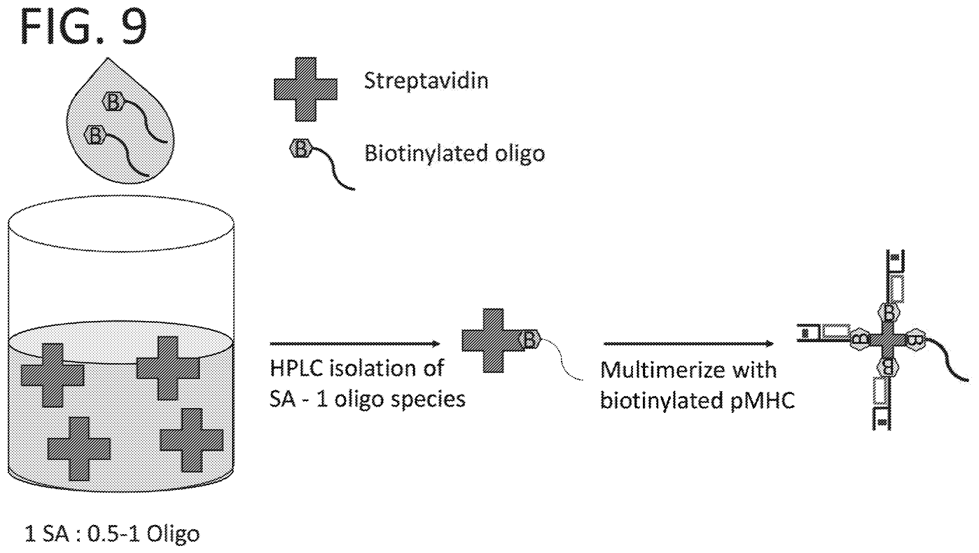

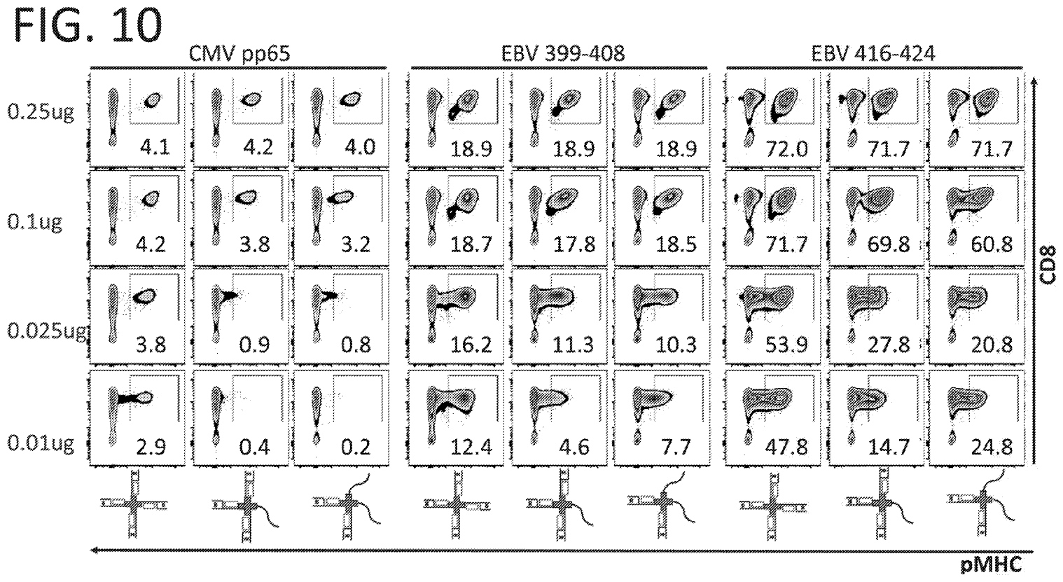

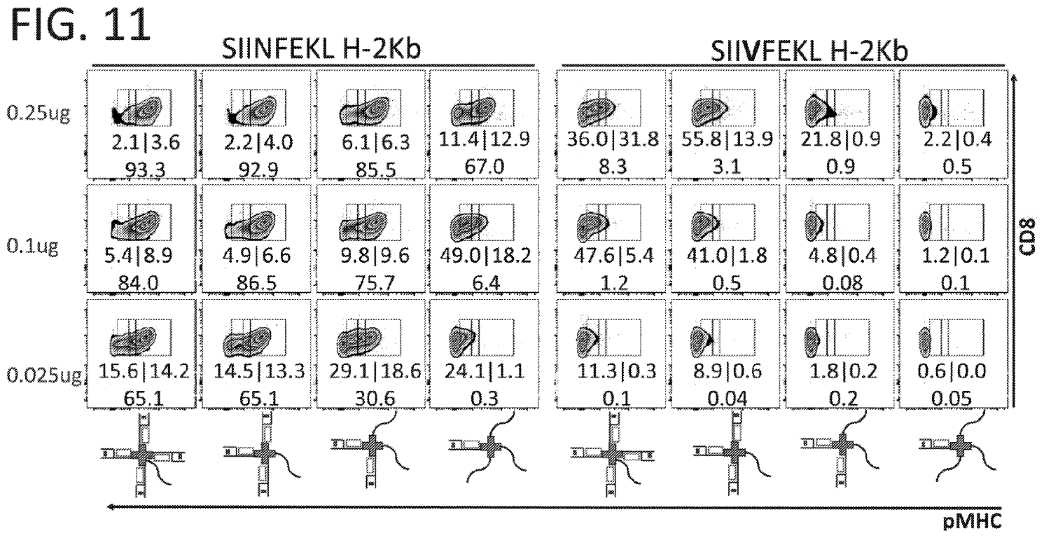

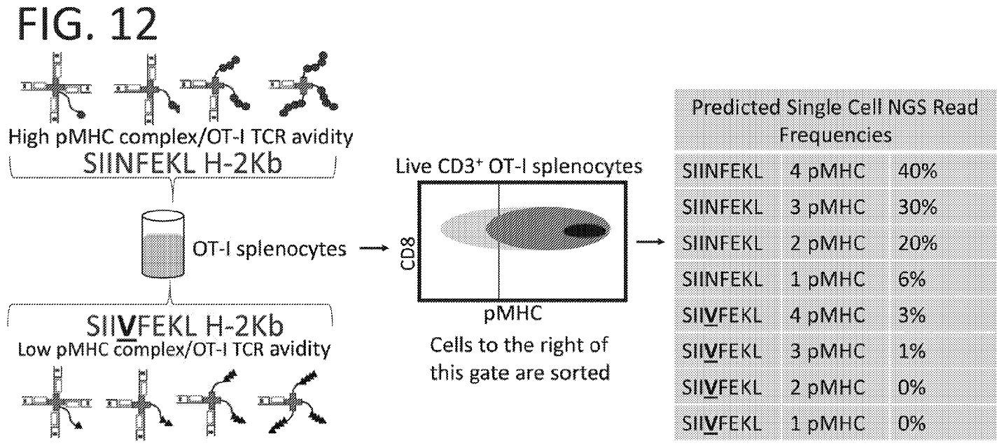

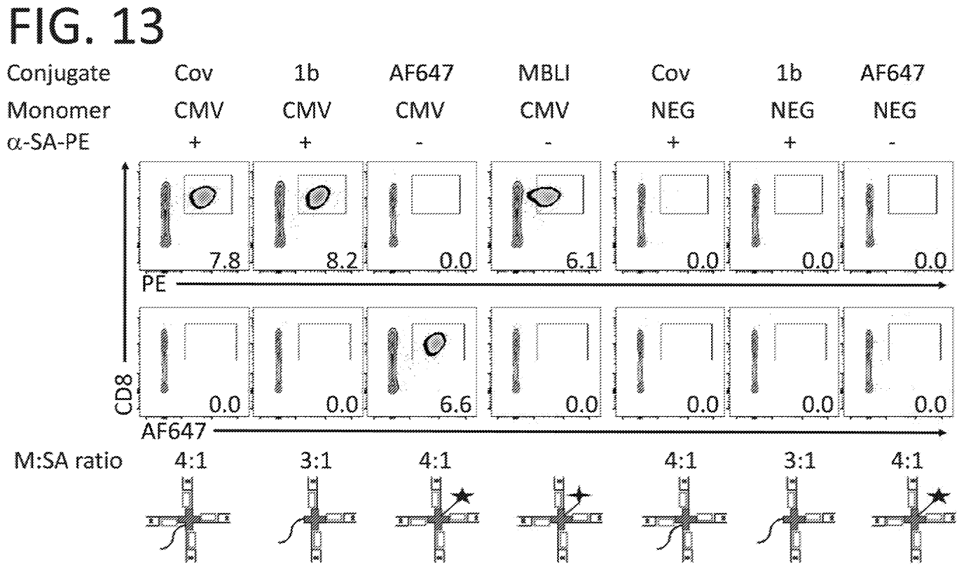

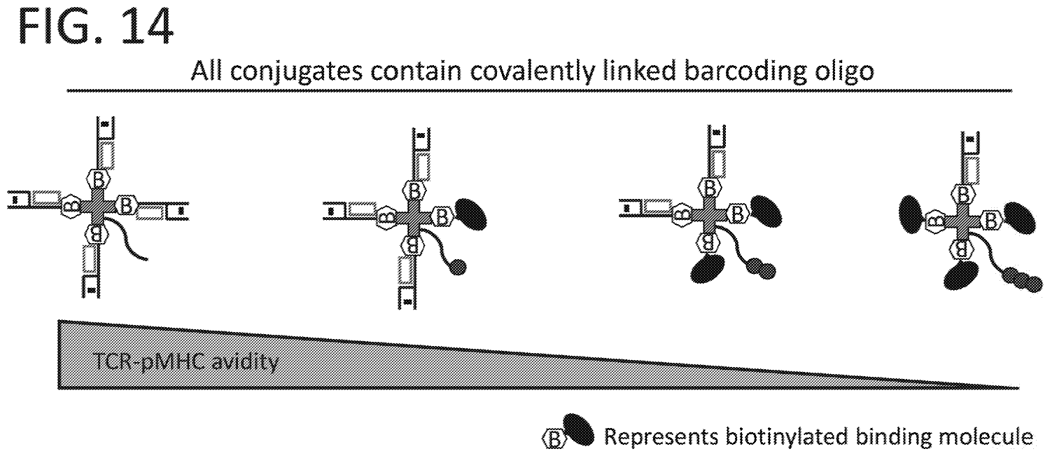

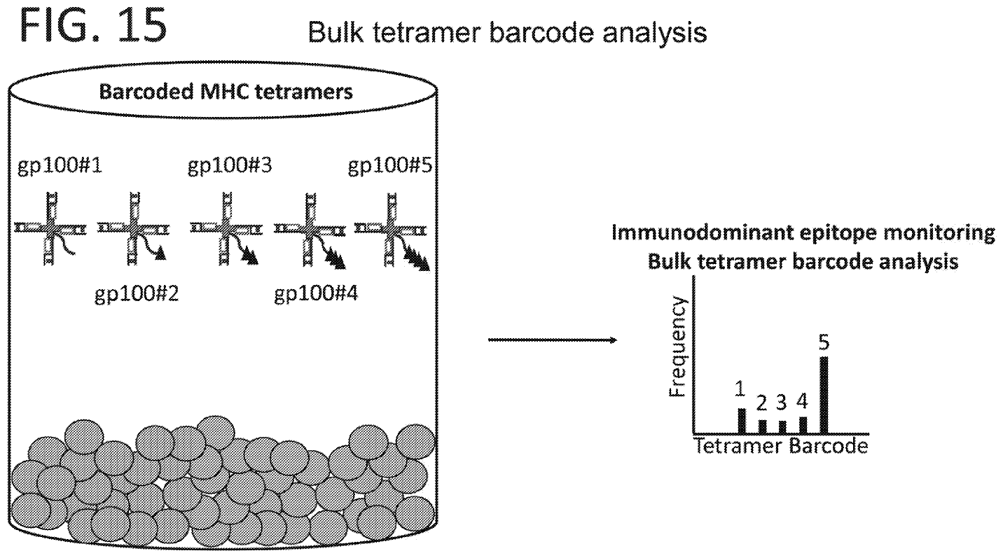

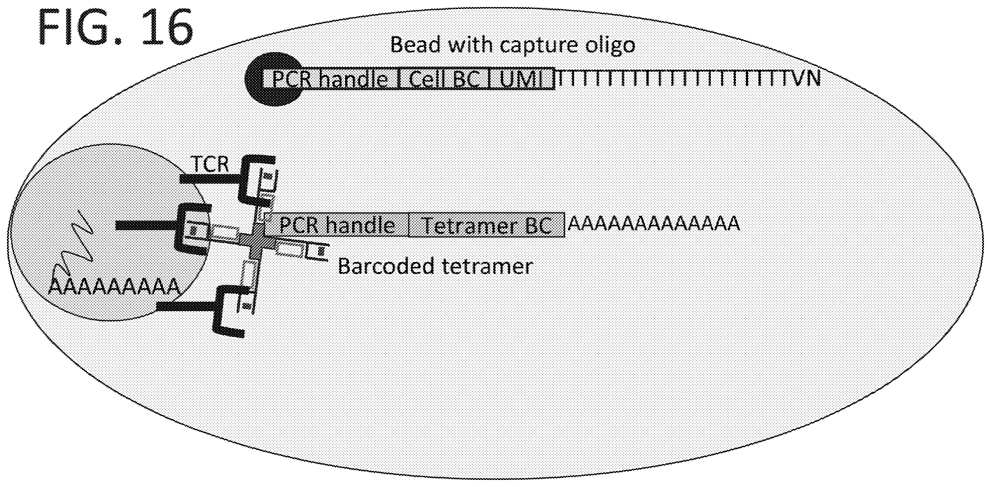

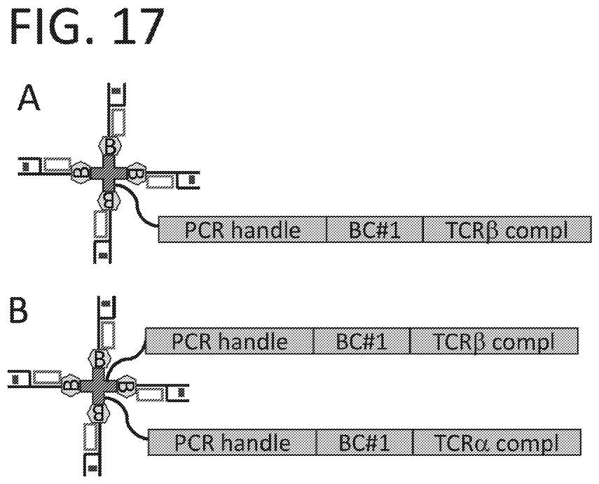

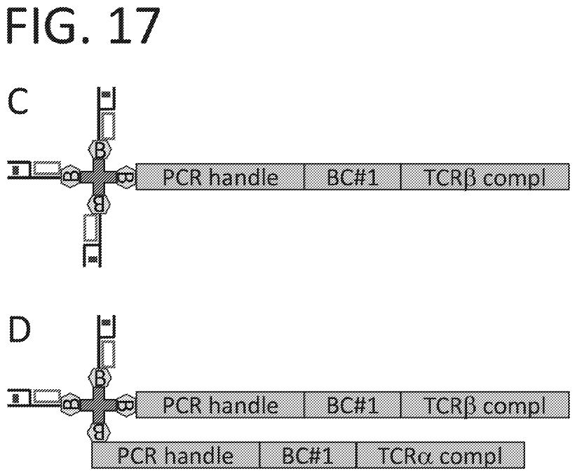

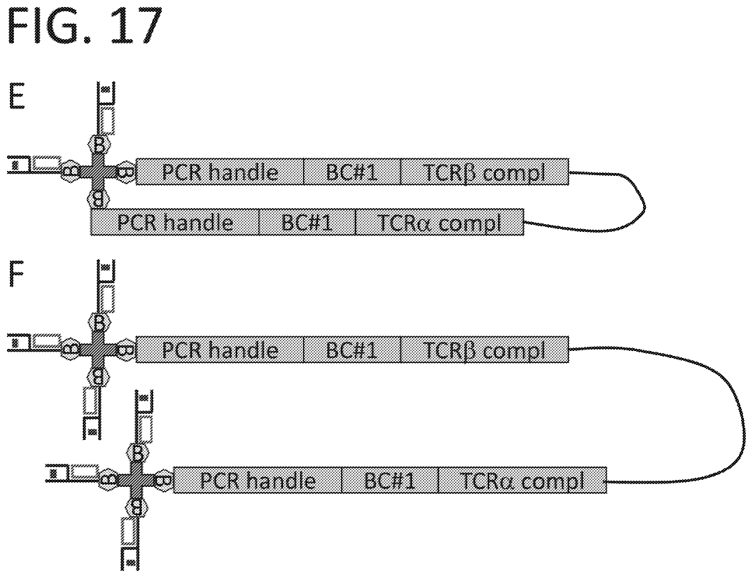

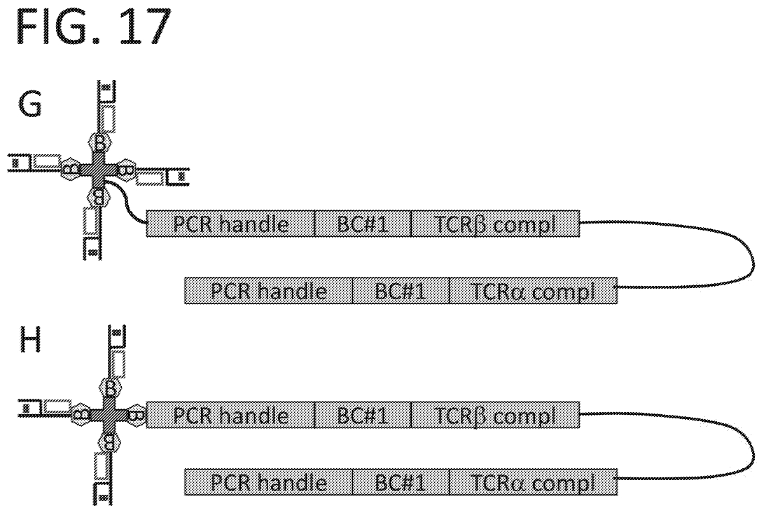

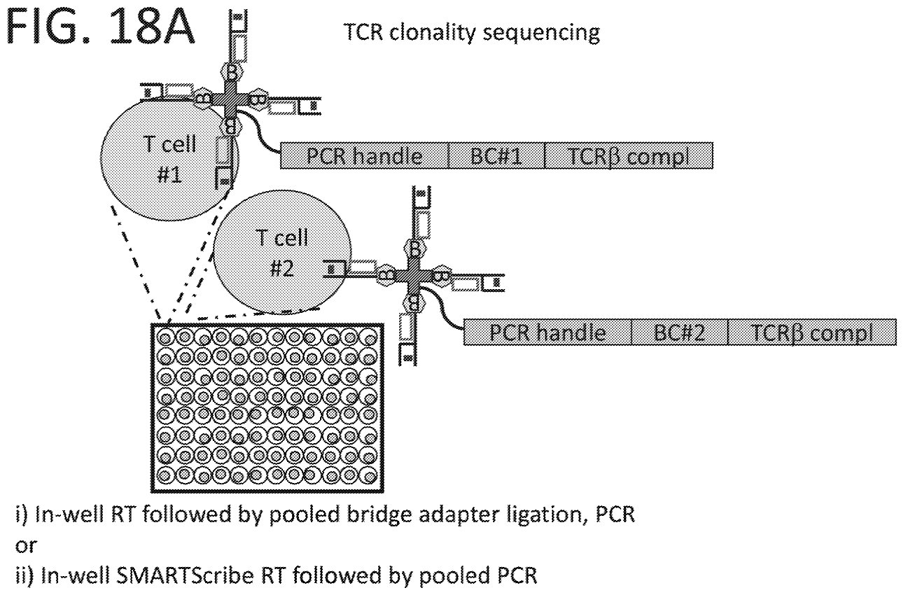

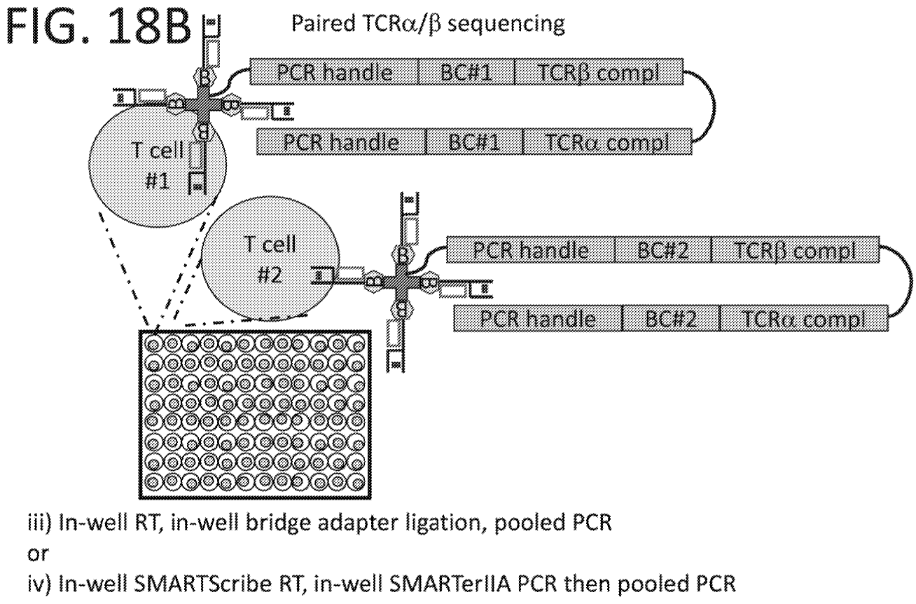

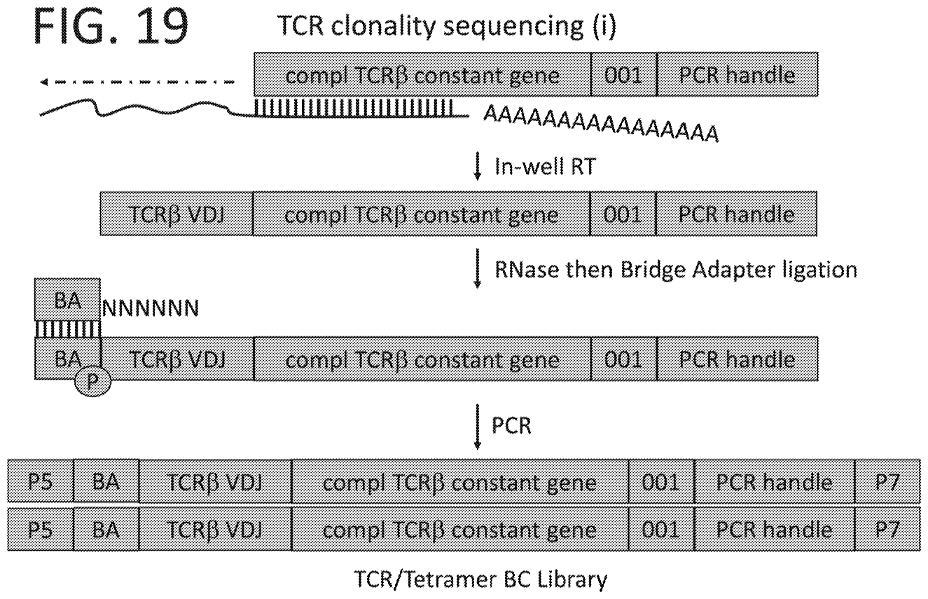

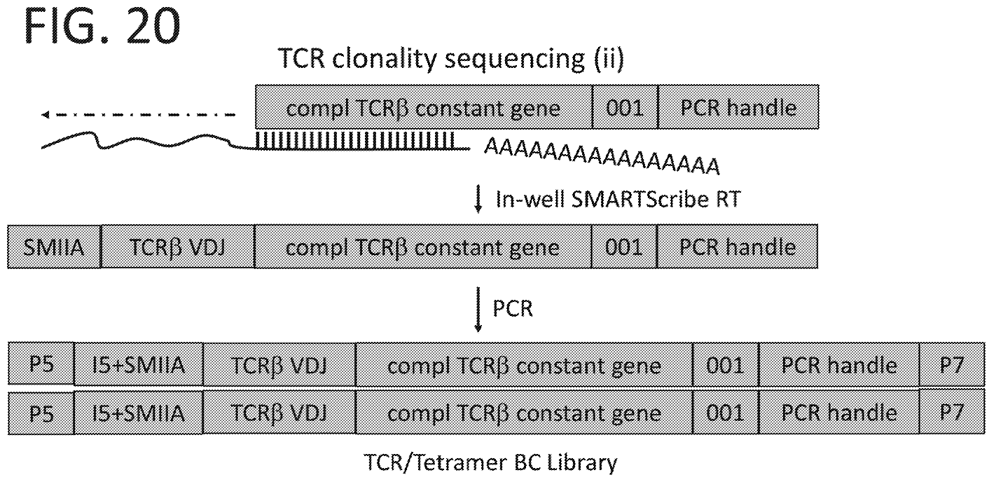

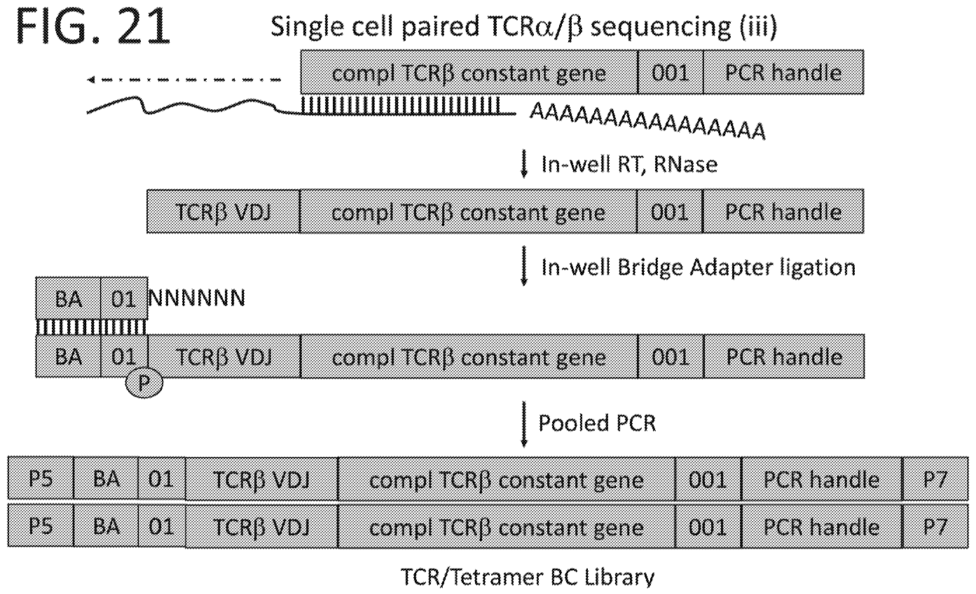

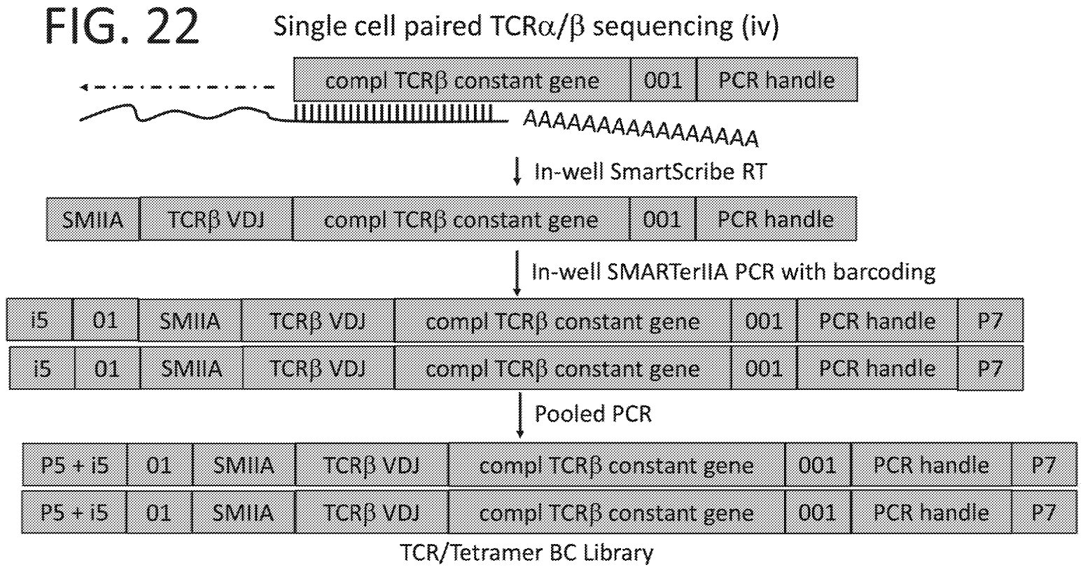

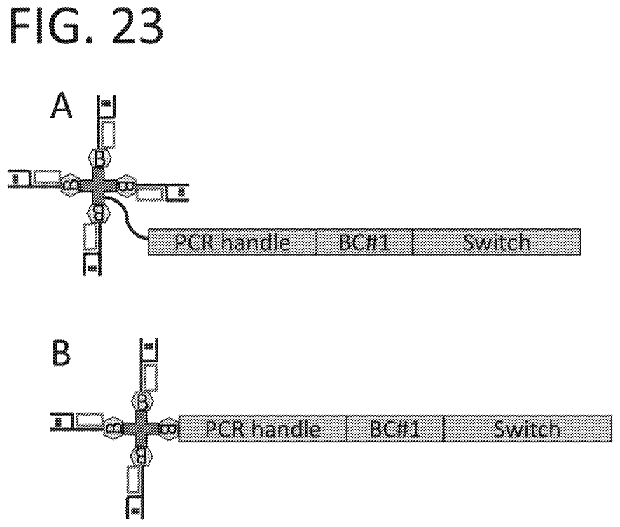

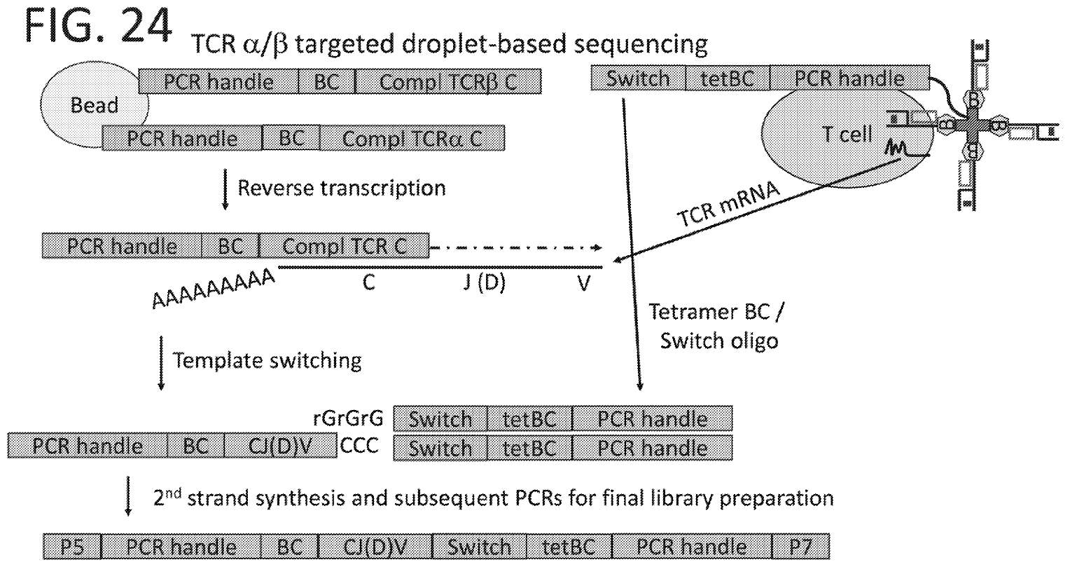

FIG. 23 contains two panels, FIG. 23A-23B, illustrating barcoded pMHC multimers containing switch oligos for droplet-based single cell TCRα/β single cell sequencing. In FIG. 23A, pMHC multimer with covalently linked oligo containing the switch sequence. In FIG. 23B, pMHC multimer with non-covalently linked oligo containing the switch sequence. Additional designs for this type of pMHC multimer include but are not limited to those described in FIG. 5 and FIG. 10.

FIG. 24 depicts the use of droplet-based sequencing beads (including but not limited to hydrogel beads, hard beads or dissolvable beads) conjugated to oligos composed of a PCR handle, cell barcode (BC) and a TCR constant gene complementary sequence. For simplicity, just one droplet is illustrated. Each bead can contain TCRα and/or TCRβ targeting ligo sequences. If both TCRα and TCRβ type oligos are used, both will contain the same cell barcode for a given bead. TCRα and/or TCRβ oligos can be dissociable by one of several means including but not limited to photocleavable bonds or disulfide bonds. In addition, each oligo can contain UMIs for PCR deduplication.

Single droplets harboring a single bead and a single cell (in this case a barcoded tetramer positive T cell is displayed) will contain lysing reagents that release both gel bead oligos, T cell mRNA as well as tetramer-positive T cell-bound barcoding oligo. pMHC tetramer bound oligo can also be made to dissociate by cleavable bonds. Reverse transcription adds TCR V(D)J sequence from TCR mRNA transcripts with a triplicate deoxycytidine stretch at the 3′ end of the bead oligo (for simplicity only a single reverse transcribed gene is depicted below the droplet). This deoxycytidine stretch binds switch oligo for template switching. Subsequent 2^{nd} strand synthesis and PCR amplifications complete library preparation.

### DETAILED DESCRIPTION OF THE INVENTION

The present disclosure describes generation of barcoded pMHC multimers compatible with flow cytometric applications including but not limited to single cell or bulk cell fluorescence-activated cell sorting (FACS) as well as compatibility with NGS-based applications, and other platforms including multiplexed analyte analysis and single cell analysis. This disclosure also describes generation of barcoded pMHC multimers through both covalent and non-covalent oligo attachment and the combination of these multimers in pMHC-TCR avidity assessment. This disclosure also describes a new pMHC multimer barcoding approach targeting TCR α/β constant genes simplifying sequencing library preparation by simultaneous processing of TCR sequences and pMHC multimer barcodes. Barcoding oligo length and/or sequence are not fixed and can be varied including usage of various base modifications.

This disclosure describes at least two oligo barcoding strategies for pMHC multimers, in some embodiments, pMHC tetramers. The first uses a barcoded poly-A tailed oligo linking a specific pMHC with corresponding T cell transcriptome, including TCR sequences. The second barcoding approach uses oligos conjugated to streptavidin that target the TCRα and/or TCRβ locus whereby both oligos harbor a tetramer barcode. Importantly, if both TCRα and TCRβ oligos are used simultaneously, they would contain the same tetramer barcoding information for a given tetramer. With this approach, all reverse-transcribed TCR sequences contain the tetramer barcode sequence negating the need for separate library preparations.

### MHC Proteins

The MHC proteins provided and used in the compositions and methods of the present disclosure may be any suitable MHC molecules known in the art where it is desirable to exchange the peptide that the MHC protein originally contained with another peptide. Generally, they have the formula (α-β-P)ₙ, where n is at least 2, for example between 2-10, e.g. 4. α is an α chain of a class I or class II MHC protein. β is a β chain, herein defined as the β chain of a class II MHC protein or β₂ microglobulin for a MHC class I protein. P is a peptide antigen.

The MHC proteins may be from any mammalian or avian species, e.g. primate sp., particularly humans; rodents, including mice, rats and hamsters; rabbits; equines, bovines, canines, felines; etc. For instance, the MHC protein may be derived from the human HLA proteins or the murine H-2 proteins. HLA proteins include the class II subunits HLA-DPa, HLA-DPβ, HLA-DQα, HLA-DQβ, HLA-DRα and HLA-DRβ, and the class I proteins HLA-A, HLA-B, HLA-C, and β2-microglobulin. H-2 proteins include the class I subunits H-2K, H-2D, H-2L, and the class II subunits I-Aα, I-Aβ, I-Eα and I-Eβ, and β2-microglobulin. Sequences of some representative MHC proteins may be found in Kabat et al. Sequences of Proteins of Immunological Interest, NIH Publication No. 91-3242, pp 724-815. MHC protein subunits suitable for use in the present invention are a soluble form of the normally membrane-bound protein, which is prepared as known in the art, for instance by deletion of the transmembrane domain and the cytoplasmic domain.

For class I proteins, the soluble form will include the α1, α2 and α3 domain. Soluble class II subunits will include the α1 and α2 domains for the a subunit, and the β1 and β2 domains for the β subunit.

The α and β subunits may be separately produced and allowed to associate in vitro to form a stable heteroduplex complex, or both of the subunits may be expressed in a single cell. Methods for producing MHC subunits are known in the art.

To prepare the MHC-peptide complex, the subunits may be combined with an antigenic peptide and allowed to fold in vitro to form a stable heterodimer complex with intrachain disulfide bonded domains. The peptide may be included in the initial folding reaction, or may be added to the empty heterodimer in a later step. In the methods of the present invention, this will be the exiting peptide. Conditions that permit folding and association of the subunits and peptide are known in the art. As one example, roughly equimolar amounts of solubilized α and β subunits may be mixed in a solution of urea. Refolding is initiated by dilution or dialysis into a buffered solution without urea. Peptides may be loaded into empty class II heterodimers at about pH 5 to 5.5 for about 1 to 3 days, followed by neutralization, concentration and buffer exchange. However, the specific folding conditions are not critical for the practice of the invention.

The monomeric complex (α-β-P) (herein monomer) may be multimerized. The resulting multimer will be stable over long periods of time. Preferably, the multimer may be formed by binding the monomers to a multivalent entity through specific attachment sites on the α or β subunit, as known in the art (e.g., as described in U.S. Pat. No. 5,635,363). The MHC proteins, in either their monomeric or multimeric forms, may also be conjugated to beads or any other support.

Frequently, the multimeric complex will be labeled, so as to be directly detectable when used in immunostaining or other methods known in the art, or will be used in conjunction with secondary labeled immunoreagents which will specifically bind the complex, as known in the art and as described herein. For example, the label may be a fluorophore, such as fluorescein isothiocyanate (FITC), rhodamine, Texas Red, phycoerythrin (PE), allophycocyanin (APC), Brilliant Violet™ 421, Brilliant UV™ 395, Brilliant Violet™ 480, Brilliant Violet™ 421 (BV421), Brilliant Blue™ 515, APC-R700, or APC-Fire750. In some embodiments, the multimeric complex is labeled by a moiety that is capable of specifically binding another moiety. For instance, the label may be biotin, streptavidin, an oligonucleotide, or a ligand. Other labels of interest may include dyes, enzymes, chemiluminescers, particles, radioisotopes, or other directly or indirectly detectable agent.

The compositions disclosed herein may comprises any suitable MHC protein. Exemplary MHC proteins and with the peptides disclosed here include H-2 Kb monomer, HLA-A*02:01 monomer, HLA-A*24:02 monomer, HLA-A*02:01 tetramer, HLA-A*24:02 tetramer, and H-2 Kb tetramer. However, any MHC allele may be used in the compositions and methods herein upon selection of an appropriate exiting peptide, according for example to known techniques for predicting the affinity of a peptide to an MHC allele.

### Definitions

The articles “a” and “an” are used herein to refer to one or to more than one (i.e., to at least one) of the grammatical object of the article. By way of example, “an element” means one element or more than one element.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The phrase “derived from” when used concerning a rearranged variable region gene “derived from” an unrearranged variable region and/or unrearranged variable region gene segments refers to the ability to trace the sequence of the rearranged variable region gene back to a set of unrearranged variable region gene segments that were rearranged to form a gene that expresses the variable domain (accounting for, where applicable, splice differences and somatic mutations). For example, a rearranged variable region gene that has undergone somatic mutation is still derived from the unrearranged variable region gene segments. In some embodiments, where the endogenous locus is replaced with a universal light chain or heavy chain locus, the term “derived from” indicates the ability to trace origin of the sequence to said rearranged locus even though the sequence may have undergone somatic mutations.

The “PCR handle” refers to a constant sequence identical to all primers, which allows PCR amplification of the barcode region described herein.

The term “barcoded region” refers to a region comprising a unique nucleotide sequence. The minimal length of this nucleotide sequence depends on the total number of MHC multimers that need to be uniquely labeled. For example, a nucleotide sequence that is 4 nucleotides long can have 256 different sequences, which can uniquely label up to 256 MHC multimers. A nucleotide sequence that is 6 nucleotides long can have 4096 different sequences, which can uniquely label up to 4096 MHC multimers. Longer tetramer sequences can be used for increased throughput.

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

Embodiment 1. A multimeric major histocompatibility complex (MHC) comprising: one or more MHC molecules linked by a backbone molecule; and at least one nucleic acid molecule linked to the backbone molecule, wherein the nucleic acid molecule comprises a central stretch of nucleic acids (barcode region), and a second stretch of nucleic acids having a sequence that displays complementarity to a target oligo.

Embodiment 2. The multimeric MHC of embodiment 1, wherein the at least one nucleic acid molecule comprises a 5′ PCR handle region, a central barcode region, and a 3′ poly-A tail region, optionally wherein the at least one nucleic acid molecule comprises a 5′ PCR handle region, a central barcode region, a unique molecular identifier (UMI), and a 3′ poly-A tail region

Embodiment 3. The multimeric MHC of embodiment 1 or 2, wherein the poly-A tail comprises at least 10 consecutive adenines.

Embodiment 4. The multimeric MHC of any one of embodiments 1-3, wherein the nucleotide of the 5′ end of the poly-A tail is G, C or T if the matching nucleotide of the target oligo is C, G or A.

Embodiment 5. The multimeric MHC of any one of embodiments 1-4, wherein the at least one nucleic acid molecule is at least 10 nucleotides in length, optionally wherein the at least one nucleic acid molecule is 10-200 nucleotides in length.

Embodiment 6. A composition comprising a plurality of subsets of multimeric MHC according to any one of embodiments 1-5, wherein each subset of multimeric MHC binds a different peptide and has a corresponding barcode region sequence.

Embodiment 7. A method of linking a specific MHC molecule with a corresponding T cell transcriptome, comprising:
a) forming a test sample comprising a plurality of multimeric MHC molecules according to any one of embodiments 1-5, T cells, and particles linked to a complementary target oligo comprising a 5′ PCR handle, a central cell barcode, a UMI, and a 3′ poly-(dT);
b) forming droplets from the test sample such that each droplet contains no more than one particle, and one T cell bound with one or more multimeric MHC molecules;
c) generating a T cell cDNA library and an MHC barcode library in each droplet; and
d) sequencing both the T cell mRNA library and the MCH barcode library, thereby linking a specific MHC molecule with the corresponding T cell transcriptome.

Embodiment 8. A multimeric MHC comprising:

one or more MHC molecules linked by a backbone molecule; and at least one nucleic acid molecule linked to said backbone, wherein said nucleic acid molecule comprises a central stretch of nucleic acids (barcode region), and a nucleotide sequence complementary to a TCR constant gene.

Embodiment 9. The multimeric MHC of embodiment 8, comprising a first type of nucleic acid molecule linked to the backbone; and wherein the first type of nucleic acid molecule comprises a central barcode region and a nucleotide sequence complementary to TCRα or TCRβ constant gene.

Embodiment 10. The multimeric MHC of embodiment 8, comprising a first and a second type of nucleic acid molecule linked to the backbone; and wherein:

the first type of nucleic acid molecule comprises a central barcode region and a nucleotide sequence complementary to TCRα constant gene;

the second type of nucleic acid molecule comprises a central barcode region and a nucleotide sequence complementary to TCRβ constant gene; and

the barcode regions of the two types of the nucleic acid molecules have the same sequence;

optionally wherein each type of the nucleic acid molecule further comprises a UMI, and the UMI sequence of the first type of nucleic acid molecule is different from the UMI sequence of the second type of nucleic acid molecule.

Embodiment 11. The multimeric MHC of any one of embodiments 8-10, wherein the nucleic acid molecule comprises a 5′ PCR handle, a central barcode region, and a 3′ nucleotide sequence complementary to a TCR constant gene.

Embodiment 12. The multimeric MHC of any one of embodiments 8-11, wherein the nucleic acid molecule comprises a nucleotide sequence complementary to the 5′ end of the TCR constant gene.

Embodiment 13. The multimeric MHC of any one of embodiments 8-12, wherein the TCR constant gene is TCRα constant gene, TCRβ constant 1 gene, or TCRβ constant 2 gene.

Embodiment 14. The multimeric MHC of any one of embodiments 8-13, wherein the 5′ end and/or the 3′ end nucleic acid molecules are linked to the backbone molecule.

Embodiment 15. The multimeric MHC of any one of embodiments 8-14, wherein the nucleic acid molecule further comprises a unique molecular identifier (UMI) adjacent to the barcode region.

Embodiment 16. The multimeric MHC of any one of embodiments 8-15, wherein the at least one nucleic acid molecule is at least 10 nucleotides in length, optionally wherein the at least one nucleic acid molecule is 10-200 nucleotides in length.

Embodiment 17. A composition comprising: a plurality of subsets of multimeric MHC according to any one of embodiments 8-16, wherein each subset of multimeric MHC binds a different peptide and has a corresponding barcode region sequence.

Embodiment 18. A method of linking a specific MHC molecule to corresponding TCRα and/or TCRβ sequences, comprising:
a) providing one or more multimeric major histocompatibility complexes according to any one of embodiments 8-16;
b) contacting said multimeric MHC molecules with T cells;
c) separating T cells bound with the multimeric MHC molecules from those that do not bind the multimeric MHC molecules;
d) lysing the separated T cells;
e) generating a DNA library wherein each DNA molecule comprises sequences of TCRα and/or TCRβ gene as well as the MHC barcode; and
f) sequencing the DNA library, thereby linking the specific MHC molecule to the corresponding TCRα and/or TCRβ sequences.

Embodiment 19. The method of embodiment 18, wherein the step c) is accomplished by FACS sorting or magnetic bead-based separation.

Embodiment 20. The method of embodiment 18 or 19, wherein the multimeric MHC molecules are directly or indirectly fluorescently labeled.

Embodiment 21. The method of any one of embodiments 18-20, wherein the T cells bound with barcoded MHC molecules are bulk sorted in a single collection tube.

Embodiment 22. The method of any one of embodiments 18-21, wherein cognate T cells bound with barcoded MHC molecules are sorted into individual plate wells as single cells.

Embodiment 23. A multimeric MHC comprising:

two or more MHC molecules linked by a backbone molecule; and

at least one nucleic acid molecule linked to said backbone, wherein said nucleic acid molecule comprises a central stretch of nucleic acids (barcode region), and a template switch oligo sequence.

Embodiment 24. The multimeric MHC of embodiment 23, wherein the nucleic acid molecule comprises a 5′ PCR handle, a central barcode region, a UMI and 3′ template switch oligo sequence.

Embodiment 25. The multimeric MHC of embodiment 23 or 24, wherein the template switch oligo sequence comprises a 3′ stretch of 3 riboguanosines.

Embodiment 26. The multimeric MHC of any one of embodiments 23-25, wherein the at least one nucleic acid molecule is at least 10 nucleotides in length, optionally wherein the at least one nucleic acid molecule is 10-200 nucleotides in length.

Embodiment 27. A composition comprising: a plurality of subsets of multimeric MHC according to any one of embodiments 23-26, wherein each subset of multimeric MHC binds a different peptide and has a corresponding barcode region sequence.

Embodiment 28. A method of linking a specific MHC molecule to corresponding TCRα and/or TCRβ sequences, comprising:
a) forming a test sample comprising a plurality of multimeric MHC molecules according to any one of embodiments 23-26, T cells, and beads conjugated to an oligo comprising a 5′ PCR handle, a central cell barcode, a UMI and a 3′ nucleotide sequence complementary to a TCR constant gene;
b) forming droplets from the test sample such that each droplet contains no more than one bead, and one T cell bound with one or more multimeric MHC molecules;
c) generating a DNA library wherein each DNA molecule comprises sequences of TCRα and/or TCRβ gene as well as the MHC barcode; and
d) sequencing the DNA library, thereby linking a specific MHC molecule to corresponding TCRα and/or TCRβ sequences.

Embodiment 29. The method of embodiment 28, wherein the bead is selected from hydrogel bead, hard bead and dissolvable bead.

Embodiment 30. The method of embodiment 28 or 29, wherein the bead is conjugated to two oligos, wherein the first oligo comprises a 5′ PCR handle, a central cell barcode, a UMI and a 3′ nucleotide sequence complementary to a TCRα constant gene; the second oligo comprises a 5′ PCR handle, a central cell barcode, a UMI and a 3′ nucleotide sequence complementary to a TCRβ constant gene; and the central cell barcodes for the two oligos have the same sequence.

Embodiment 31. The method of any one of embodiments 28-30, wherein the DNA library generation step c) comprises reverse transcription of TCR mRNA using MMLV reverse transcriptase.

Embodiment 32. The multimeric MHC or method of any one of the preceding embodiments, wherein the PCR handle enables the library preparation of the barcode sequence.

Embodiment 33. The multimeric MHC or method of any one of the preceding embodiments, wherein the PCR handle has the i7 adapter sequence.

Embodiment 34. The multimeric MHC or method of any one of the preceding embodiments, wherein the barcode region comprises at least 4 nucleotides.

Embodiment 35. The multimeric MHC or method of any one of the preceding embodiments, wherein the barcode region comprises 6 nucleotides.

Embodiment 36. The multimeric MHC or method of any one of the preceding embodiments, wherein the backbone molecule is selected from the group consisting of polysaccharides, glucans, dextran, streptavidin, and a streptamer multimer.

Embodiment 37. The multimeric MHC or method of any one of the preceding embodiments, wherein the MHC molecule is linked to the backbone via a streptavidin-biotin binding, via the MHC heavy chain, or via the MHC light chain (β2M).

Embodiment 38. The multimeric MHC or method of any one of the preceding embodiments, wherein the MHC molecule is linked to the backbone via a streptavidin-biotin binding.

Embodiment 39. The multimeric MHC or method of any one of the preceding embodiments, wherein the multimeric MHC comprises at least four MHC molecules.

Embodiment 40. The multimeric MHC or method of any one of the preceding embodiments, wherein the at least one nucleic acid molecule further comprises chemical modifications.

Embodiment 41. The multimeric MHC or method of any one of the preceding embodiments, wherein the 5′ or 3′ end of the at least one nucleic acid molecule is attached to an amino group via a spacer.

Embodiment 42. The multimeric MHC or method of any one of the preceding embodiments, wherein the spacer is a 6 carbon spacer or a 12 carbon spacer.

Embodiment 43. The multimeric MHC or method of any one of the preceding embodiments, wherein the at least one nucleic acid molecule comprises phosphorothioated nucleotides at the 5′ end and/or 3′ end.

Embodiment 44. The multimeric MHC or method of any one of the preceding embodiments, wherein the linkage between the at least one nucleic acid molecule and the backbone molecule allow for inducible dissociation of the nucleic acid molecules.

Embodiment 45. The multimeric MHC or method of any one of the preceding embodiments, wherein the at least one nucleic acid molecule is linked to the backbone molecule via a disulfide bridge.

Embodiment 46. The multimeric MHC or method of any one of the preceding embodiments, wherein the disulfide bridge is formed by combine the succinimidyl-6-hydrazino-nicotinamide (S-HyNic) modified backbone molecule with the succinimidyl-4-formylbenzamide analog (S-SS-4FB) modified nucleic acid molecule that is 5′-amino-modified.

Embodiment 47. The multimeric MHC or method of any one of the preceding embodiments, wherein the at least one nucleic acid molecule is linked to the backbone molecule via photocleavable linkages.

Embodiment 48. The multimeric MHC or method of any one of the preceding embodiments, wherein the MHC molecule is MHC class I and/or MHC class II monomer.

Embodiment 49. The multimeric MHC or method of any one of the preceding embodiments, wherein the MHC molecule is complexed with a peptide.

Embodiment 50. The multimeric MHC or method of any one of the preceding embodiments, wherein the MHC molecule is biotinylated.

Embodiment 51. The multimeric MHC or method of any one of the preceding embodiments, wherein the backbone further comprises one or more labels selected from the group consisting of fluorescent labels, His-tags, and metal-ion tags.

Embodiment 52. The multimeric MHC or method of any one of the preceding embodiments, wherein the backbone is directly conjugated with the fluorescent labels.

Embodiment 53. The multimeric MHC or method of any one of the preceding embodiments, wherein the fluorescent label is modified with 4FB and conjugated with a S-HyNic-modified backbone.

Embodiment 54. The multimeric MHC or method of any one of the preceding embodiments, wherein the fluorescent label is a fluorophore-tagged oligo.

Embodiment 55. The multimeric MHC or method of any one of the preceding embodiments, wherein the fluorophore-tagged oligo has a 5′-amino or 3′-amino modification and further modified with 4FB and conjugated with a S-HyNic-modified backbone.

Embodiment 56. The multimeric MHC or method of any one of the preceding embodiments, wherein the fluorophore-tagged oligo is complementary to the nucleic acid molecule linked to the backbone.

Embodiment 57. The multimeric MHC or method of any one of the preceding embodiments, wherein the fluorophore-tagged oligo is 10 nucleotides in length.

Embodiment 58. The multimeric MHC or method of any one of the preceding embodiments, wherein the backbone is labeled with a fluorophore labeled anti-streptavidin antibody.

Embodiment 59. The multimeric MHC or method of any one of the preceding embodiments, wherein the fluorophore is a fluorescent dye or quantum dot.

Embodiment 60. The multimeric MHC or method of any one of the preceding embodiments, wherein the at least one nucleic acid molecule comprises a nucleic acid molecule selected from the group consisting of DNA, RNA, artificial nucleotides, PNA, and LNA.

Embodiment 61. The multimeric MHC or method of any one of the preceding embodiments, wherein the multimeric MHC binds cognate T cells.

Embodiment 62. The multimeric MHC or method of any one of the preceding embodiments, wherein the multimeric MHC is compatible with flow cytometric applications.

Embodiment 63. The multimeric MHC or method of any one of the preceding embodiments, wherein the flow cytometric application is single cell or bulk cell fluorescence-activated cell sorting (FACS).

Embodiment 64. The multimeric MHC or method of any one of the preceding embodiments, wherein the multimeric MHC is compatible with NGS-based applications.

Embodiment 65. The multimeric MHC or method of any one of the preceding embodiments, wherein the NGS-based application is droplet-based single cell sequencing.

Embodiment 66. A method for detecting antigen responsive cells in a sample comprising:
a) providing one or more multimeric major histocompatibility complexes according to any one of embodiments 1-5, 8-16, 23-26 and 32-65;
b) contacting said multimeric MHC molecules with said sample; and
c) detecting binding of the multimeric MHC molecules to said antigen responsive cells, thereby detecting cells responsive to an antigen present in the MHC molecules, wherein said binding is detected by amplifying the barcode region of said nucleic acid molecule linked to the one or more MHC molecules through the backbone molecule.

Embodiment 67. The method of embodiment 66, wherein the sample is selected from the group consisting of blood sample, a peripheral blood sample, a blood derived sample, a tissue sample, a body fluid, spinal fluid, and saliva.

Embodiment 68. The method of embodiment 66 or 67, wherein the sample is obtained from a mammal.

Embodiment 69. The method of any one of embodiments 66-68, wherein the method further comprises cell selection by a method selected from the group consisting of flow cytometry, FACS, magnetic-bead based selection, size-exclusion, gradient centrifugation, column attachment, and gel-filtration.

Embodiment 70. The method of any one of embodiments 66-69, wherein the amplification is PCR.

Embodiment 71. The method of any one of embodiments 66-70, wherein the detection of barcode regions of the nucleic acid molecule includes sequencing of the barcode regions or detection of the barcode region by qPCR.

### EXAMPLES

The invention now being generally described, it will be more readily understood by reference to the following examples which are included merely for purposes of illustration of certain aspects and embodiments of the present invention, and are not intended to limit the invention.

### Example 1: Construction and Experimental Validation of Barcoded pMHC Multimer Species

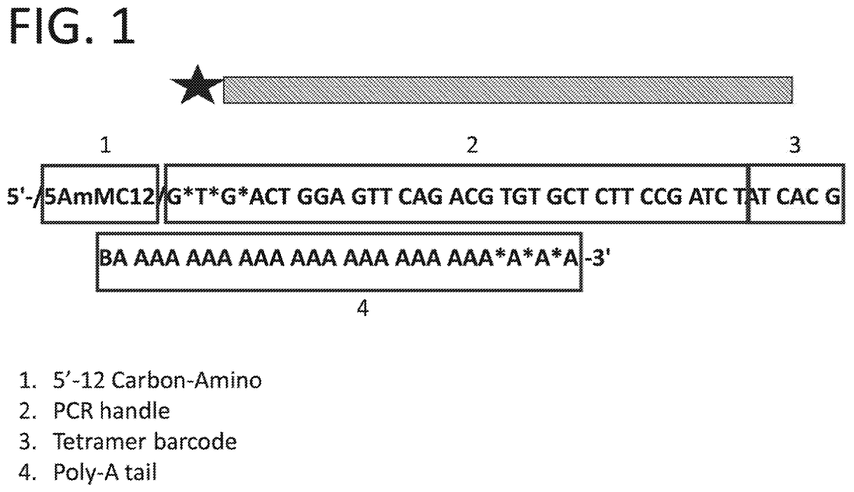

The following describes both covalent and non-covalent conjugation of oligo consisting of any sequence. For covalent conjugation, oligo (e.g. in FIG. 1) is conjugated to streptavidin through the formation of a thioether bond, although other chemistries and bond formations can be employed. In this particular instance, covalent linkage would bridge both oligo and streptavidin to a common heterobifunctional linker yielding a streptavidin-oligo conjugate (FIG. 5). Non-covalently conjugated oligo to streptavidin can be accomplished by mixing biotinylated oligo with streptavidin at optimum ratios and HPLC purification of desired streptavidin-oligo conjugate species (FIG. 5 and FIG. 9). Covalent and non-covalently derived streptavidin-oligo conjugate species can subsequently be subjected to pMHC multimerization (FIG. 5). These pMHC multimer species can be used in different combinations for pMHC-TCR avidity studies (as described in FIG. 11). Additionally, pMHC multimers of a single subtype (e.g. only pMHC trimers) can be used singularly in bulk or single cell sequencing platforms if it is known that target pMHC-TCR avidity is high enough such that the difference between 3 and 4 monomers per streptavidin will not affect TCR detection. The advantage of just using non-covalently derived streptavidin-oligo conjugates is the cost savings of forgoing chemical conjugation as well as using less monomers per streptavidin.

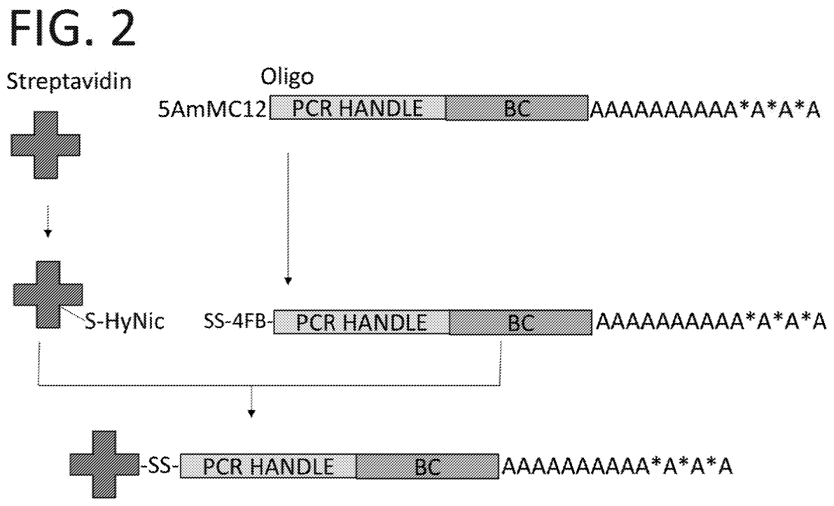

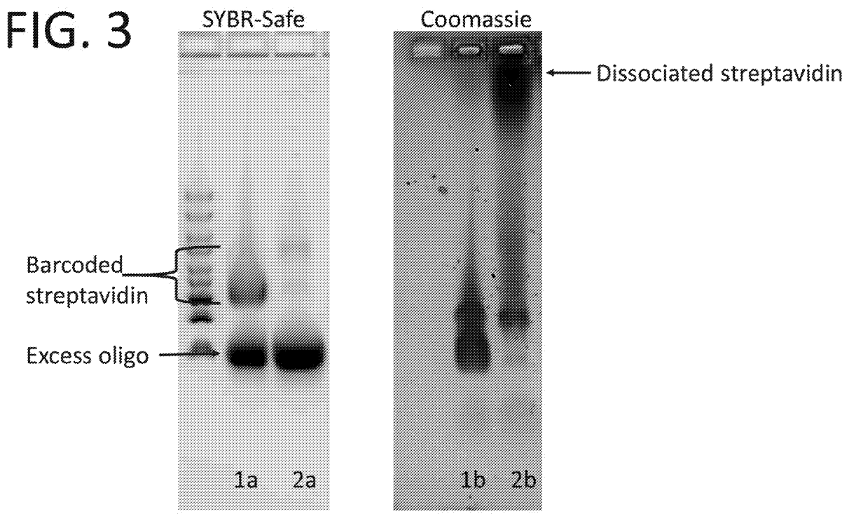

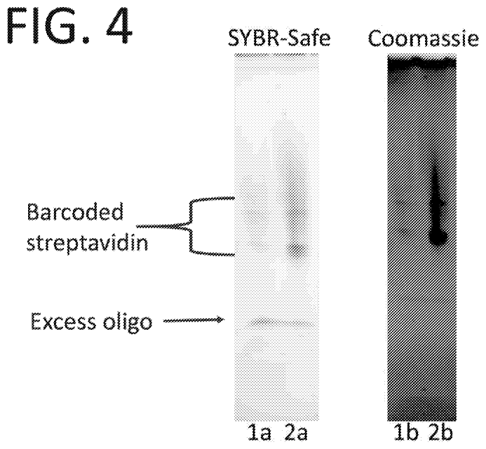

As another example, the Solulink Protein-Oligonucleotide Conjugation Kit with supplementary S-SS-4FB can be used. Streptavidin was first modified with S-HyNic. 5′-amino-modified oligo was modified with S-SS-4FB. Modified oligo and modified streptavidin were then combined to produce a directly barcoded streptavidin (FIG. 2). Alternative conjugation chemistry approaches and material providers can be used that conjugate barcoding oligo to protein and allow for inducible barcoding oligo dissociation including but not limited to photocleavable linkages. Experiments validated the ability to temporally dissociate oligo from streptavidin, in this case under reducing conditions (FIG. 3), as well as the ability of barcoded streptavidin to bind biotin (FIG. 4).

### Example 2: Fluorophore Based Tracking of Barcoded Tetramers

Barcoded tetramers can be combined with various fluorophore tagging strategies (FIGS. 6A-6C) for use in single cell and bulk cell sorting applications.

### Example 3: TCR Targeting Barcoded Tetramers

Barcoded pMHC multimers can be made to target the TCRα and/or TCRβ constant genes (FIG. 17) using the same conjugation chemistry described. This disclosure benefits scientists interested in only obtaining TCR sequences and matching pMHC information. The major advantage of this method is that only one library preparation is needed because both reverse-transcribed TCRα and/or TCRβ sequences contain the pMHC multimer barcode. Thus, a singular sequencing read will contain both the TCR sequence and pMHC identity information. This contrasts with poly-A tailed or other capture sequence-based library preparation methods, whereby the smaller pMHC barcode/antibody barcode library is processed separately and later combined with mRNA-derived libraries at the time of sequencing¹⁻⁴. The TCR targeted tetramer described in this disclosure can be combined with fluorophore detection for use in single or bulk cell sorting (FIGS. 6A-6C). Single cell sorting allows for TCR clonality studies (FIGS. 18-20), as well as potential linking of TCRα and TCRβ paired sequences to the sorted cell of origin (FIGS. 18, 21, 22).

### Example 4: Custom TCR Droplet-Based Sequencing

In another embodiment, barcoded pMHC multimers can be made such that pMHC multimer-conjugated oligo contains a template switch oligo sequence along with the pMHC multimer barcode sequence and a PCR handle sequence (FIG. 23). Key to this is that the switch oligo sequence contains a 3′ stretch of 3 riboguanosines to bind the deoxycytidines added by MMLV reverse transcriptase. In this scenario, droplet-based beads (including but not limited to hydrogel beads, hard beads or dissolvable beads) would be custom conjugated to TCRα and/or TCRβ constant gene complementary oligos with the typical cell barcode and PCR handle sequences. UMIs can be included in any of the oligo sequences for PCR deduplication. Beads with both TCRα and TCRβ targeting oligos would contain the same barcode for a given bead. Within a single droplet that contains both bead and pMHC multimer positive T cell, reverse transcription with MMLV reverse transcriptase would extend the bead-based oligo with V(D)J sequence from TCR mRNA as well as include template switching oligo sequence harboring the pMHC multimer barcode and PCR handle. Subsequent secondary strand synthesis and PCR amplification would complete library preparation for sequencing. Oligo sequence, length and modifications (including but not limited to use of locked nucleic acid bases) for both bead oligo and pMHC multimer barcoding/template switching oligo can be changed. This embodiment can be compatible with other reverse transcriptase derivatives.

This system has several key advantages. One is that only TCR sequences and pMHC barcode sequences are obtained negating the need for sequencing the whole transcriptome. Another advantage is that only one library preparation is needed since both tetramer barcode sequence and TCR sequence are on the same transcript. Another advantage is that if both TCRα and TCRβ bead oligos are used, then both are automatically paired due to the unique cell barcode sequences inside each droplet. Lastly, because only pMHC multimer positive T cells have the template switch/pMHC multimer barcoded oligo which contains one of the two PCR handles, only pMHC multimer positive T cells contribute to sequencing libraries.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.
1. Bentzen, A. K., et al., Large-scale detection of antigen-specific T cells using peptide-MHC-I multimers labeled with DNA barcodes. Nat Biotechnol, 2016. 34(10): p. 1037-1045.
2. Stoeckius, M., et al., Simultaneous epitope and transcriptome measurement in single cells. Nat Methods, 2017. 14(9): p. 865-868.
3. Peterson, V. M., et al., Multiplexed quantification of proteins and transcripts in single cells. Nat Biotechnol, 2017. 35(10): p. 936-939.
4. Zhang, S. Q., et al., High-throughput determination of the antigen specificities of T cell receptors in single cells. Nat Biotechnol, 2018.
5. Dahotre, S. N., et al., DNA-Barcoded pMHC Tetramers for Detection of Single Antigen-Specific T Cells by Digital PCR. Anal Chem, 2019. 91(4): p. 2695-2700.
6. Altman, J. D. and M. M. Davis, MHC-Peptide Tetramers to Visualize Antigen-Specific T Cells. Curr Protoc Immunol, 2016. 115: p. 17 3 1-17 3 44.
7. Krogsgaard, M. AAI 2019, Abstract 194.58.
8. Krummey, S. M., et al., Low-Affinity Memory CD8+ T Cells Mediate Robust Heterologous Immunity. J Immunol, 2016. 196(6): p. 2838-46.
9. Zhou, Z. X., et al., Mapping genomic hotspots of DNA damage by a single-strand-DNA-compatible and strand-specific ChIP-seq method. Genome Res, 2013. 23(4): p. 705-15.

### EQUIVALENTS

Those skilled in the art will recognize, or be able to ascertain using no more than routine experimentation, many equivalents to the specific embodiments encompassed by the present invention described herein. Such equivalents are intended to be encompassed by the following claims.

1. A peptide-Major Histocompatibility Complex (pMHC) barcoded multimer comprising:
at least one tunable pMHC entity, wherein said pMHC entity comprises:
at least one pMHC molecule linked by a backbone molecule: and
at least one nucleic acid molecule per backbone molecule,
wherein said nucleic acid molecule comprises a covalently or non-covalently linked conjugate.

2. The pMHC multimer of claim 1, wherein the nucleic acid molecule comprises:
a central stretch of pMHC barcoding nucleotides, and
a second stretch of nucleotides with complementarity to a target oligo.

3. (canceled)

4. The pMHC multimer of claim 1, wherein the backbone molecule is a streptavidin, and wherein the pMHC molecule is linked to the backbone via a streptavidin-biotin binding.

5. The pMHC multimer of claim 1, wherein the multimer comprises at least one, at least two, at least three, or at least four pMHC entities per backbone molecule.

6. (canceled)

7. The pMHC multimer of claim 1, wherein the streptavidin is:
(i) covalently conjugated to the at least one nucleic acid molecule, thereby providing at least four MHC monomers per streptavidin; or
(ii) non-covalently conjugated to the at least one nucleic acid molecule, wherein the nucleic acid molecule is biotinylated, and the at least one biotinylated nucleic acid molecule and streptavidin is complexed in a ratio, wherein the ratio is selected from the group consisting of:
1 streptavidin:1 oligo,
1 streptavidin:2 oligos, and
1 streptavidin:3 oligos.

8.-9. (canceled)

10. The pMHC multimer of claim 1, wherein the streptavidin is covalently conjugated to the at least one nucleic acid molecule, wherein the nucleic acid molecule comprises a barcode and at least one biotin binding site, wherein said binding site comprises:
biotinylated peptides, biotinylated proteins, biotinylated polymers, biotinylated fluorophores, biotinylated cleavable oligos, or biotinylated agents.

11. The pMHC multimer of claim 1, wherein the at least one nucleic acid molecule comprises a 5′ PCR handle region, a central barcode region, optionally a UMI, or optionally a 3′ poly-A tail region of at least 10 consecutive adenines.

12-13. (canceled)

14. A composition comprising a plurality of subsets of the pMHC multimer according to claim 1, wherein each subset of pMHC multimer binds a different peptide and has a corresponding barcode region sequence.

15. A method of linking a specific MHC molecule with a corresponding T cell transcriptome, comprising:
a) forming a test sample comprising a plurality of the pMHC multimer molecules according to claim 1, T cells, and particles linked to a binding target oligo comprising a 5′ PCR handle, a central cell barcode, UMI, and a bait sequence;
b) forming droplets from the test sample such that each droplet contains no more than one particle, and one T cell bound with one or more pMHC multimer molecules;
c) generating a T cell cDNA library and an pMHC barcode library in each droplet; and
d) sequencing both the T cell mRNA library and the MCH barcode library, thereby linking a specific MHC molecule with the corresponding T cell transcriptome.

16. The method of claim 15, wherein the bait sequence is 3′ poly-(dT).

17. A multimeric pMHC comprising:
one or more pMHC molecules linked by a backbone molecule; and
at least one nucleic acid molecule linked to said backbone, wherein said nucleic acid molecule comprises a central stretch of nucleic acids (barcode region) designed to be amplified and a nucleotide sequence complimentary to a TCR constant gene.

18. (canceled)

19. The multimeric pMHC of claim 17, comprising a first and a second type of nucleic acid molecule linked to the backbone; and wherein:
the first type of nucleic acid molecule comprises a central barcode region and a nucleotide sequence complimentary to TCRα constant gene, and
the second type of nucleic acid molecule comprises a central barcode region and a nucleotide sequence complimentary to TCRβ constant gene; and
the barcode regions of the two types of the nucleic acid molecules have the same sequence; and
optionally the UMI sequences for each of the two types of the nucleic acid molecules would be random and thus different from each other although they would be the located in the same region of the respective nucleic acids.

20. The multimeric pMHC of claim 17, wherein the nucleic acid molecule comprises a 5′ PCR handle, a central barcode region, UMI and a 3′ nucleotide sequence complimentary to a TCR constant gene.

21. The multimeric pMHC of claim 17, wherein the nucleic acid molecule comprises a nucleotide sequence complimentary to the 5′ end of the TCR constant gene.

22. The multimeric MHC of claim 17, wherein the TCR constant gene is TCRα constant gene, TCRβ constant 1 gene, or TCRβ constant 2 gene.

23-25. (canceled)

26. A composition comprising: a plurality of subsets of multimeric pMHC according to claim 17, wherein each subset of multimeric MHC binds a different peptide and has a corresponding barcode region sequence.

27. A method of linking a specific MHC molecule to a corresponding TCRα and/or TCRβ sequences, comprising:
a) providing one or more multimeric pMHC according to claim 17;
b) contacting said multimeric pMHC molecules with T cells;
c) separating T cells bound with the multimeric MHC molecules from those that do not bind the multimeric MHC molecules;
d) lysing the separated T cells;
e) generating a DNA library wherein each DNA molecule comprises a sequence of TCRα and/or TCRβ gene as well as the pMHC barcode; and
f) Sequencing the DNA library, thereby linking the specific pMHC molecule to the corresponding TCRα and/or TCRβ sequences.

28. The method of claim 27, wherein the step c) is accomplished by FACS sorting or magnetic bead-based separation.

29. (canceled)

30. The method of claim 27, wherein the T cells bound with barcoded pMHC molecules are bulk sorted in a single collection tube, or cognate T cells bound with barcoded pMHC molecules are sorted into individual plate wells as single cells.

31. (canceled)

32. A multimeric pMHC comprising:
one or more pMHC molecules linked by a backbone molecule; and
at least one nucleic acid molecule linked to said backbone, wherein said nucleic acid molecule comprises a central stretch of nucleic acids (barcode region) designed to be amplified, and a template switch oligo sequence.

33. The multimeric pMHC of claim 32, wherein the nucleic acid molecule comprises a 5′ PCR handle, a central barcode region, a UMI and 3′ template switch oligo sequence.

34. The multimeric pMHC of claim 32, wherein the template switch oligo sequence comprises a 3′ stretch of 3 riboguanosines.

35. (canceled)

36. A composition comprising: a plurality of subsets of multimeric pMHC according to claim 32, wherein each subset of multimeric pMHC binds a different peptide and has a corresponding barcode region sequence.

37. A method of linking a specific pMHC molecule to a corresponding TCRα and/or TCRβ complementary sequences, comprising:
a) forming a test sample comprising a plurality of multimeric pMHC molecules according to claim 32, T cells, and beads conjugated to an oligo comprising a 5′ PCR handle, a central cell barcode, UMI and a 3′ nucleotide sequence complementary to a TCR constant gene;
b) forming droplets from the test sample such that each droplet contains no more than one bead, and one T cell bound with one or more multimeric MHC molecules;
c) generating a DNA library wherein each DNA molecule comprises a sequence of TCRα and/or TCRβ gene as well as the pMHC barcode; and
d) sequencing the DNA library, thereby linking a specific pMHC molecule to a corresponding TCRα and/or TCRβ sequences.

38. (canceled)

39. The method of claim 37, wherein the bead is conjugated to two oligos, wherein the first oligo comprises a 5′ PCR handle, a central cell barcode, UMI and a 3′ nucleotide sequence complementary to a TCRα constant gene; the second oligo comprises a 5′ PCR handle, a central cell barcode, UMI and a 3′ nucleotide sequence complementary to a TCRβ constant gene; and the central cell barcodes for the two oligos have the same sequence.

40. The method of claim 37, wherein the DNA library generation step c) comprises reverse transcription of TCR mRNA using MMLV reverse transcriptase.

41-46. (canceled)

47. The multimeric pMHC of claim 1, wherein the at least one nucleic acid molecule further comprises chemical modifications.

48-55. (canceled)

56. The multimeric pMHC of claim 1, wherein the MHC molecule is MHC class I and/or MHC class II monomer.

57-58. (canceled)

59. The multimeric pMHC of claim 1, wherein the backbone further comprises one or more labels selected from the group consisting of fluorescent labels, His-tags, and metal-ion tags.

60-70. (canceled)

71. A method for detecting antigen responsive cells in a sample comprising:
a) providing one or more multimeric pMHC according to claim 1;
b) contacting said multimeric pMHC molecules with said sample; and
c) detecting binding of the multimeric pMHC molecules to said antigen responsive cells, thereby detecting cells responsive to an antigen present in the MHC molecules, wherein said binding is detected by amplifying the barcode region of said nucleic acid molecule linked to the one or more MHC molecules through the backbone molecule.

72. The method of claim 71, wherein the sample is selected from the group consisting of blood sample, a peripheral blood sample, a blood derived sample, a tissue sample, a body fluid, spinal fluid, and saliva.

73. (canceled)

74. The method of claim 71, wherein the method further comprises cell selection by a method selected from the group consisting of flow cytometry, FACS, magnetic-bead based selection, size-exclusion, gradient centrifugation, column attachment, and gel-filtration.

75. (canceled)

76. The method of claim 71, wherein the detection of barcode regions of the nucleic acid molecule includes sequencing of the barcode regions or detection of the barcode region by qPCR.
